(19) United States
(12) Patent Application Publication
(10) Pub. No.: US 2026/0060570 A1
(43) Pub. Date: Mar. 5, 2026

(54) SYSTEM AND METHOD FOR BODY MOTOR FUNCTION ASSESSMENT

(71) Applicant: UNIVERSITY OF VIRGINIA PATENT FOUNDATION, Charlottesville, VA (US)

(72) Inventors: Laura BARNES, Earlysville, VA (US); Rebecca SCHARF, Charlottesville, VA (US); Mehdi BOUKHECHBA, Charlottesville, VA (US); Silvia S. BLEMKER, Charlottesville, VA (US); Robert GUTIERREZ, Bristow, VA (US); Allison MCCRADY, Lancaster, OH (US)

(21) Appl. No.: 19/108,271

(22) PCT Filed: Sep. 1, 2023
(86) PCT No.: PCT/US23/31902

§ 371 Date: Mar. 3, 2025

Related U.S. Application Data
(60) Provisional application No. 63/403,688, filed on Sep. 2, 2022.

(51) Int. Cl. A61B 5/11; A61B 5/00; A61B 8/00

(52) U.S. Cl. CPC A61B 5/1107; A61B 5/4842; A61B 5/7264; A61B 5/7275; A61B 8/4494; A61B 2562/0219

(57) ABSTRACT

A computerized system calculates a composite index for sets of functional movement scores that quantify motor function abilities for patients. Wearable sensors, kinetic energy sensors, ultrasound imagers, and force sensors are used to store the composite index from predictors of the functional movement scores, the predictors comprising a respective shape of motion factor, a respective motion symmetry factor, and a respective motion speed factor for each of the sets of functional movement scores. The software tabulates the respective composite index by calculating a probability that the respective shape of motion factor, the respective motion symmetry factor, and the respective motion speed factor correspond to one of the sets of functional movement scores.

### CROSS-REFERENCE TO RELATED APPLICATION

This application claims priority to and benefit of U.S. Provisional Patent Application Ser. No. 63/403,688, entitled “System and Method for Body Motor Function Assessment” filed Sep. 2, 2022, which is hereby incorporated by reference herein in its entirety as if fully set forth below.

### STATEMENT OF GOVERNMENT INTEREST

This disclosure was made with government support under Grant No. AR069393, awarded by the National Institutes for Health. The government has certain rights in the invention.

### FIELD

This disclosure relates to creating a scoring system to evaluate motor function and muscle activity in patients diagnosed with disabling diseases.

### BACKGROUND

Neuromuscular disorders, such as (SMA) and Duchenne Muscular Dystrophy (DMD), cause progressive muscular degeneration and loss of motor function for 1 in 6,000 children. Traditional upper limb motor function assessments do not quantitatively measure patient-performed motions, which makes it difficult to track progress for incremental changes. Assessing motor function in children with neuromuscular disorders is particularly challenging because they can be nervous or excited during experiments, or simply be too young to follow precise instructions. These challenges translate to confounding factors such as performing different parts of the arm curl slower or faster (phase variability) which affects the assessed motion quality.

While there has been significant progress in the development of therapeutic treatments for two prominent neuromuscular disorders, Spinal Muscular Atrophy (SMA) and Duchenne Muscular Dystrophy (DMD), the assessments for measuring patient progress have remained stagnant [9]. SMA affects approximately 1 in 11,000 babies born each year, with a life expectancy at just under 2 years while DMD affects 1 in 3,500 males. These new therapeutics have increased the life expectancy of patients with SMA from infants to young adults, and from early teens to mid-thirties for patients with DMD. For boys with DMD, they typically lose their ability to walk in late childhood to early teen years and have limited use of their arms after their early teens.

Currently, there are several motor function assessments that physicians use to measure a patient's functional abilities within a clinical setting, such as the Children's Hospital of Philadelphia Infant Test of Neuromuscular Disorders (CHOP-INTEND) [6], the Brooke Upper Extremity Scale [5], and the Hammersmith Functional Motor Scale Expanded (HFMSE) [10]. Unfortunately, all the assessments previously mentioned use outcomes based on ordinal, subjective rating scales, which are unable to account for incremental changes, for both quantity and quality of movement. Another problem with data collected through clinical visits is related to sparsity. Due to the nature of the neuromuscular disorder, patient visits are infrequent, usually every 4-6 months. Thus, the low sampling frequency makes it challenging to gauge the temporal dynamics of the neuromuscular disorder.

Pediatric motion quality assessment is challenging because of several confounding factors, depending upon the modality used. Firstly, translations of the device (e.g., moving the kinect/camera closer or further away from the subject) can affect the trajectory representations based on spatial positions. Similarly, rotations of either the participant or the sensor can change representations based on (x,y,z) coordinates [1]. Variations in limb length affect all representations based on 3d-position. Finally, phase variation (for instance, choosing to do the first half of the curl slower than the last) tends to affect data collected from wearable devices [3].

With emerging therapies in neuromuscular disorders, monitoring progress is vital: Spinal Muscular Atrophy (SMA) is the leading genetic cause of death in infants, with a life expectancy of less than 2 years, which until recently, had no treatment for this disease. The development of Nusinersen, an antisense oligonucleotide therapy to treat SMA, and other forms of treatment, such as gene therapy, have led to a historic moment in the care of patients with SMA, showing progress not currently captured with current motor scale tests [2]-[5]. Duchenne Muscular Dystrophy (DMD) is the most common muscular dystrophy, and the most common genetic cause of death in boys. Duchenne affects about one in 5,000 boys and young men, leading to progressive muscle weakness, causing loss of ambulation, and then loss of arm function, followed by respiratory weakness and cardiomyopathy. Currently, new treatments are being developed for Duchenne, but these treatments have been slow to progress through clinical trials and to reach patients, in part due to lack of sensitive and specific markers of improvement which can be used to monitor effectiveness. As with any medical treatment, the outcomes vary from patient to patient. Therefore, new research must be done to measure the effectiveness of interventions (or lack thereof) and motor change over in neuromuscular disorders, from micro-movements to muscular regeneration, in a group of patients who previously didn't live long enough to keep measures of progress. While there have been several works focused on other movement disorders (e.g., Parkinson's, Multiple Sclerosis) and specifically on characterizing gait, this is the first body of work to our knowledge targeting SMA and DMD with the specific goal of measuring/quantifying microvariations in upper extremities. [6]-[8]. However, this work also has applicability to a large spectrum of disease processes including Cerebral Palsy, Alzheimer, Myelomeningocele, Brain Injury, Stroke, and other neuromotor conditions.

### SUMMARY

Other aspects and features according to the example embodiments of the present disclosure will become apparent to those of ordinary skill in the art, upon reviewing the following detailed description in conjunction with the accompanying figures.

In an implementation, a computerized system calculates a respective composite index for respective sets of functional movement scores that quantify motor function abilities for patients diagnosed with a disease. A wearable sensor is positioned proximately to a test patient's anatomy to gather test motion shape data and test motion symmetry data. A respective wearable sensor is positioned proximately to a control patient's anatomy to gather control motion shape data and control motion symmetry data. A kinetic energy sensor is positioned proximately to a test patient's anatomy to gather test kinetic energy data. A respective control kinetic energy sensor positioned proximately to a control patient's anatomy to gather control kinetic energy data. A computer stores software implemented by a computer processor in communication with computer memory, wherein the computer has access to the test motion shape data, the test motion symmetry data, the control motion shape data, the control motion symmetry data, the test kinetic energy data, the control kinetic energy data, and wherein the software executes computerized steps with the computer processor to calculate and store the composite index from predictors of the functional movement scores. The predictors include a respective shape of motion factor, a respective motion symmetry factor, and a respective motion speed factor for each of the sets of functional movement scores. The computerized steps include calculating the respective shape of motion factor for each of the test motion shape data and the control motion shape data; calculating the respective motion symmetry factor for each of the test motion symmetry data and the control motion symmetry data; calculating the respective motion speed factor for each of the test kinetic energy data and the control kinetic energy data; and using the software to tabulate the respective composite index by calculating a probability that the respective shape of motion factor, the respective motion symmetry factor, and the respective motion speed factor correspond to one of the sets of functional movement scores.

In another implementation, calculating the probability includes assigning weights to the predictors and running the predictors through a regression analysis using machine learning software.

In another implementation, the system further includes an imaging device producing images of muscles of the test patient during motion assessment exercises and a dynamometer gathering measurements of force exerted by the muscles during the motion assessment exercise, wherein the computer receives the images and the dynamometer measurements and classifies corresponding muscle scores for the muscles, wherein the software uses the corresponding muscle scores as an additional predictor in tabulating the composite index.

In another implementation, the imaging device includes a linear array ultrasound probe that images a transverse plane and a longitudinal plane of the muscles.

In another implementation, the images include muscle measurements having anatomical cross sectional area (ACSA), muscle thickness, and tissue echogenicity.

In another implementation, the computer classifies corresponding muscle scores by calculating an anatomical cross sectional area (ACSA) of the muscles and dividing the ACSA by an average echogenicity of the muscle.

In another implementation, the wearable sensor and the respective wearable sensor each comprise a multimodal accelerometer.

In another implementation, the wearable sensor and the respective wearable sensor each further include e a gyroscope.

In another implementation a wearable sensor and the respective wearable sensor each include a wireless sensor in electronic communication with the computer.

In another implementation, the kinetic energy sensor and the respective control kinetic energy sensor each include a manometer measuring forearm pronation and bicep curl motion.

In another implementation, a video camera records video and audio data of the test patient during motion assessment exercises.

In another implementation, a computer implemented method assesses body movements performed by a test patient diagnosed with a disease, and the method includes gathering test motion data for the test patient with a sensor positioned proximately to the test patient's anatomy. The method further includes gathering control motion data for a control patient with a respective sensor positioned proximately to the control patient's anatomy and storing, in computer memory connected to a computer processor, test motion data trajectories and control motion data trajectories for respective sets of the test motion data and the control motion data. The method also includes using software accessible by the computer processor and the computer memory to perform computer automated steps including aligning the control motion data trajectories in the time domain; calculating a mean motion data trajectory for the control motion data trajectories; aligning the test motion data trajectories to the mean motion data trajectory in the time domain; and computing distance measurements between the test motion data trajectories and the mean motion data trajectory to quantify severity of motor function symptoms of the disease in the test patient.

In another implementation, computing distances includes computing at least one of an amplitude distance, a phase distance, or a cosine distance between the test motion data trajectories and the mean motion data trajectories.

In another implementation, computing distances includes computing a phase distance by computing a test motion data warping function to align the test motion data trajectories to the mean motion data trajectory.

In another implementation, the method includes, prior to aligning the control motion data trajectories, calculating the square root velocity function of the test motion data trajectories and the control motion data trajectories.

In another implementation, the method includes computing the distance measurements in the time domain across a range of values for the square root velocity functions of the test motion data trajectories to the mean motion data trajectory.

In another implementation, the computer implemented method of claim 12, further comprising using the software to complete phase amplitude separation on the test motion data trajectories and the control motion trajectories prior to calculating the mean motion data trajectory.

In another implementation, gathering the test motion data and the control motion data includes attaching the sensor to the test patient's anatomy and attaching the respective sensor to the control patient's anatomy.

In another implementation gathering the test motion data and the control motion data includes gathering data with a camera.

In another implementation, the method includes gathering muscle structure image data from the test patient and correlating the muscle structure image data with the distance measurements in the time domain.

In another implementation, the method includes gathering muscle structure data with an ultrasound image.

In another implementation, the method includes gathering the test motion data and the control motion data by wearing the sensor or the respective sensor on the body of a test patient or a control patient.

In another implementation, a computerized system calculates a respective composite index for sets of functional movement scores that quantify motor function abilities for patients diagnosed with a disease. A wearable sensor is positioned proximately to a test patient's anatomy to gather test motion shape data and test motion symmetry data. A respective wearable sensor is positioned proximately to a control patient's anatomy to gather control motion shape data and control motion symmetry data. A kinetic energy sensor is positioned proximately to a test patient's anatomy to gather test kinetic energy data. A respective control kinetic energy sensor positioned proximately to a control patient's anatomy to gather control kinetic energy data. A computer includes software implemented by a computer processor in communication with computer memory, wherein the computer has access to the test motion shape data, the test motion symmetry data, the control motion shape data, the control motion symmetry data, the test kinetic energy data, the control kinetic energy data, and the software executes computerized steps with the computer processor to calculate and store the composite index from predictors of the functional movement scores. The predictors include a respective shape of motion factor, a respective motion symmetry factor, and a respective motion speed factor for each of the sets of functional movement scores. The software implements steps of calculating the respective shape of motion factor for each of the test motion shape data and the control motion shape data; calculating the respective motion symmetry factor for each of the test motion symmetry data and the control motion symmetry data; calculating the respective motion speed factor for each of the test kinetic energy data and the control kinetic energy data; and using the software to tabulate the respective composite index by calculating a probability that the respective shape of motion factor, the respective motion symmetry factor, and the respective motion speed factor correspond to one of the sets of functional movement scores, wherein the shape of motion factor quantifies a severity of motor function symptoms of the disease in the test patient, and wherein the shape of motion factor is calculated according to a computer implemented method.

The shape of the motion factor is calculated by assessing body movements performed by a test patient diagnosed with a disease, and the method includes gathering test motion data for the test patient with a sensor positioned proximately to the test patient's anatomy. The method further includes gathering control motion data for a control patient with a respective sensor positioned proximately to the control patient's anatomy and storing, in computer memory connected to a computer processor, test motion data trajectories and control motion data trajectories for respective sets of the test motion data and the control motion data. The method also includes using software accessible by the computer processor and the computer memory to perform computer automated steps including aligning the control motion data trajectories in the time domain; calculating a mean motion data trajectory for the control motion data trajectories; aligning the test motion data trajectories to the mean motion data trajectory in the time domain; and computing distance measurements between the test motion data trajectories and the mean motion data trajectory to quantify severity of motor function symptoms of the disease in the test patient.

In another implementation, an imaging device producing images of muscles of the test patient during motion assessment exercises; and a dynamometer gathers measurements of force exerted by the muscles during the motion assessment exercise, wherein the computer receives the images and the dynamometer measurements and classifies corresponding muscle scores for the muscles; and wherein the software uses the corresponding muscle scores as an additional predictor in tabulating the composite index.

In another implementation, gathering the test motion data and the control motion data includes wearing the sensor or the respective sensor on the body of a test patient or a control patient.

### BRIEF DESCRIPTION OF THE DRAWINGS

Reference will now be made to the accompanying drawings, which are not necessarily drawn to scale.

FIG. 1 illustrates graphical trajectories of data gathered according to embodiments of this disclosure in a top row and a bottom row. The top row shows a set of healthy trajectories used to find a set of temporal warping functions which align the trajectories with each other, allowing one to find the elastic mean for healthy cohort. The bottom row shows aligning patient movement trajectories for DMD+SMA trajectories to the elastic mean of healthy cohort. The warping functions corresponding to DMD+SMA are more distorted than the ones corresponding to healthy cohorts. Based on the healthy elastic mean, these trajectories are used to compute three distances: amplitude (based on difference in y values), phase: based on differences in warping functions, and cosine: based on cosine similarity between the aligned functions. The x axis represent samples in the time domain and the y axis represent the square root velocity functions of the samples.

FIG. 2 illustrates box plots corresponding to amplitude, phase and cosine distances. The box plots show a t-test with unequal variances, comparing healthy cohort with DMD+SMA. In all three distances, DMD+SMA distances are statistically different from the healthy ones.

FIG. 3 illustrates distance matrices pre and post performing curve registration. The registered curves seem to display a much nicer block structure. Several patients of the DMD+SMA cohort perform motion on par with healthy. Participant 34 performs motion much worse than other healthy patients. Amplitude distance extracted from the registered curves is shown to be linearly related with muscle score captured via handheld dynamometer. The phase distance is related to Brooke's score, the current gold standard for functional assessment.

FIG. 4 is a sequence of motion quality assessments for identified patients over different phases of the arm curls plotted against a mean value. The x axis illustrates sample numbers in the time domain and the y axis shows rolling correlations of square root velocity functions for the distance values described herein.

FIG. 5 is a basic, schematic representation of an ultrasound system 700 according to an aspect of an embodiment of the present invention that is referred to in order to generally describe the operations of an ultrasound system to produce an image of an object.

FIG. 6 is a block diagram illustrating an example of a computer in the form of a machine upon which one or more aspects of embodiments of the present disclosure can be implemented.

FIG. 7 shows an experiment setup having an example of a Microsoft Kinect V2 camera and two MbientLab MMR+sensors, which are placed on the top of each hand

FIG. 8 illustrates graphical trajectories of data gathered according to embodiments of this disclosure in a top row and a bottom row. In the top row, given a set of healthy trajectories, this disclosure finds a set of temporal warping functions which align the trajectories with each other, allowing one to find a mean shape for the healthy cohort. Bottom row shows aligning DMD+SMA trajectories to the mean shape of the healthy cohort. The warping functions corresponding to DMD+SMA are more distorted than the ones corresponding to Healthy cohorts. Deviations from the healthy mean shape are used to compute two distances: amplitude (based on difference in y values) and phase (based on differences in warping functions). The x axis represent samples in the time domain and the y axis represents the square root velocity functions of the samples.

FIG. 9 represents distribution of functional scores based on amplitude (right) and phase (left) distances that are statistically different between cohorts. DMD: children with Duchenne muscular dystrophy and SMA: children with spinal muscular atrophy and Healthy: typically developing age/sex matched controls.

FIG. 10A illustrates an echogenicity measurement in longitudinal plane, identifying a region of interest in a patient's muscle according to this disclosure and provides measurements and data from the ultrasound measurements of the biceps muscle.

FIG. 10B illustrates a cross sectional area and echogenicity measurements in transverse plane.

FIG. 10C illustrates a maximum voluntary elbow torque dynamometry setup to be used with ultrasound image acquisition.

FIG. 10D illustrates a power law relationship between estimated specific tension and average echogenicity. DMD: children with Duchenne muscular dystrophy and SMA: children with spinal muscular atrophy and Healthy: typically developing age/sex matched controls.

FIG. 11 illustrates an amplitude score that is linearly related with the muscle score which was captured via imaging device such as an ultrasound.

FIG. 12 illustrates data relating the muscle score to the functional score over time (6 months apart). Arrows indicate the direction from the first visit to the second visit. DMD: children with Duchenne muscular dystrophy and SMA: children with spinal muscular atrophy.

FIG. 13 illustrates clinical functional scoring for patients according to a Brooke's scoring protocol and shows limited recognition of change in motor functions in a patient.

FIG. 14 illustrates clinical functional scoring for patients according to a CHOP INTEND scoring protocol and shows limited recognition of change in motor functions in a patient.

FIG. 15 shows the sensors, images, and force measurement devices used to plot muscle score data as discussed herein.

FIG. 16 shows muscle scores for numerous patients according to this disclosure.

FIG. 17A illustrates the results of a visual predictive check (VPC) for kinetic energy of motion gathered from patients with sensors including but not limited to a manometer. VPC1 includes a loss of angular speed dimension as disclosed herein.

FIG. 17B illustrates an example of a test set up for a patient with sensors attached to or proximate a patient's body and a video camera gathering image data.

FIG. 17C illustrates an example of a test set up for another patient with sensors attached to or proximate a patient's body and a video camera gathering image data.

FIG. 18A illustrates the results of a visual predictive check (VPC) for motion asymmetry data gathered from patients with sensors including but not limited to wearable devices having gyroscopes and accelerometers therein. VPC3 includes a loss of symmetry in a motor function test according to this disclosure.

FIG. 18B illustrates an example of a test set up for a patient with sensors attached to or proximate a patient's body and a video camera gathering image data.

FIG. 18C illustrates an example of a test set up for another patient with sensors attached to or proximate a patient's body and a video camera gathering image data.

FIG. 19 is a graphical representation of an example of generating a composite index of functional movement scoring by combining three dimensions or predictors according to aspects of this disclosure (for instance via regression based approaches like proportional hazard models or machine learning approaches)

FIG. 20A illustrates how curve registration for motor function tests paired with age as a variable, according to embodiments of this disclosure, allow for aligning data trajectories and illustrating how cohorts tend to be similar during testing.

FIG. 20B illustrates how curve registration for motor function tests paired with maximum score value as a variable, according to embodiments of this disclosure, allow for aligning data trajectories and illustrating how cohorts tend to be similar during testing.

FIG. 20C illustrates a trajectory for amplitude distance, indicating deviation in angular velocity from a reference trajectory, according to this disclosure for three different cohorts.

FIG. 20D illustrates a trajectory for cosine distance, indicating trajectory shape similarity, according to this disclosure for three different cohorts.

FIG. 20E illustrates raw data for the cosine distance of FIG. 20D.

FIG. 21 illustrates cross correlation between wearable features and clinical measurements using images gathered from imaging devices, including but not limited to ultrasound equipment.

### DETAILED DESCRIPTION

Although example embodiments of the present disclosure are explained in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

It must also be noted that, as used in the specification and the appended claims, the singular forms “a.” “an” and “the” include plural referents unless the context clearly dictates otherwise. Ranges may be expressed herein as from “about” or “approximately” one particular value and/or to “about” or “approximately” another particular value. When such a range is expressed, other exemplary embodiments include from the one particular value and/or to the other particular value.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

As discussed herein, a “subject” (or “patient”) may be any applicable human, animal, or other organism, living or dead, or other biological or molecular structure or chemical environment, and may relate to particular components of the subject, for instance specific organs, tissues, or fluids of a subject, may be in a particular location of the subject, referred to herein as an “area of interest” or a “region of interest.”

Some references, which may include various patents, patent applications, and publications, are cited in reference lists and discussed in the disclosure provided herein. The citation and/or discussion of such references is provided merely to clarify the description of the present disclosure and is not an admission that any such reference is “prior art” to any aspects of the present disclosure described herein. In terms of notation, “[n]” corresponds to the n^{th} reference in the list. For example, “[3]” refers to the 3rd reference in the list, namely Zhang, K., Zuo, W., Chen, Y., Meng, D., Zhang, L.: Beyond a Gaussian Denoiser: Residual Learning of Deep CNN for Image Denoising. IEEE Transactions on Image Processing. 26, 3142-3155 (2017). All references cited and discussed in this specification are incorporated herein by reference in their entireties and to the same extent as if each reference was individually incorporated by reference.

A detailed description of aspects of the present disclosure, in accordance with various example embodiments, will now be provided with reference to the accompanying drawings. The drawings form a part hereof and show, by way of illustration, specific embodiments and examples. In referring to the drawings, like numerals represent like elements throughout the several figures. Some experimental data are presented herein for purposes of illustration and should not be construed as limiting the scope of the present disclosure in any way or excluding any alternative or additional embodiments.

It should be appreciated that any number and type of computer-based medical imaging systems or components, including various types of commercially available medical imaging systems and components, may be used to practice certain aspects of the present disclosure. Systems as described herein with respect to example embodiments are not intended to be specifically limited to ultrasound or magnetic resonance imaging (MRI) implementations or the particular system shown in the figures.

One or more data acquisition or data collection steps as described herein in accordance with one or more embodiments may include acquiring, collecting, receiving, or otherwise obtaining data such as imaging data corresponding to an area of interest. By way of example, data acquisition or collection may include acquiring data via a data acquisition device, receiving data from an on-site or off-site data acquisition device or from another data collection, storage, or processing device. Similarly, data acquisition or data collection devices of a system in accordance with one or more embodiments of the present disclosure may include any device configured to acquire, collect, or otherwise obtain data, or to receive data from a data acquisition device within the system, an independent data acquisition device located on-site or off-site, or another data collection, storage, or processing device.

By way of example, and not limitation, computer-storage media (also referred to herein as a “computer-readable storage medium” or “computer-readable storage media”) may include volatile and non-volatile, removable and non-removable media implemented in any method or technology for storage of information such as computer-storage instructions, data structures, program modules, or other data. For example, computer storage media includes, but is not limited to, RAM, ROM, EPROM, EEPROM, flash memory or other solid state memory technology, CD-ROM, digital versatile disks (“DVD”), HD-DVD, BLU-RAY, or other optical storage, magnetic cassettes, magnetic tape, magnetic disk storage or other magnetic storage devices, or any other medium which can be used to store the desired information and which can be accessed by the computer Transitory signals are not “computer-storage media”, “computer-readable storage medium” or “computer-readable storage media” as described herein.

According to various embodiments, the computer may operate in a networked environment using connections to other local or remote computers through a network via a network interface unit connected to the bus. The network interface unit may facilitate connection of the computing device inputs and outputs to one or more suitable networks and/or connections such as a local area network (LAN), a wide area network (WAN), the Internet, a cellular network, a radio frequency network, a Bluetooth-enabled network, a Wi-Fi enabled network, a satellite-based network, or other wired and/or wireless networks for communication with external devices and/or systems. The computer may also include an input/output controller for receiving and processing input from a number of input devices. Input devices may include one or more of keyboards, mice, stylus, touchscreens, microphones, audio capturing devices, or image/video capturing devices. An end user may utilize such input devices to interact with a user interface, for example a graphical user interface, for managing various functions performed by the computer 200.

The bus may enable the processing unit to read code and/or data to/from the mass storage device or other computer-storage media. The computer-storage media may represent apparatus in the form of storage elements that are implemented using any suitable technology, including but not limited to semiconductors, magnetic materials, optics, or the like. The computer-storage media may represent memory components, whether characterized as RAM, ROM, flash, or other types of technology. The computer-storage media may also represent secondary storage, whether implemented as hard drives or otherwise. Hard drive implementations may be characterized as solid state or may include rotating media storing magnetically-encoded information. The program modules, which include the imaging application, may include instructions that, when loaded into the processing unit and executed, cause the computer to provide functions associated with embodiments illustrated herein. The program modules may also provide various tools or techniques by which the computer may participate within the overall systems or operating environments using the components, flows, and data structures discussed throughout this description.

In general, the program modules may, when loaded into the processing unit and executed, transform the processing unit and the overall computer from a general-purpose computing system into a special-purpose computing system. The processing unit may be constructed from any number of transistors or other discrete circuit elements, which may individually or collectively assume any number of states. More specifically, the processing unit may operate as a finite-state machine, in response to executable instructions contained within the program modules. These computer-executable instructions may transform the processing unit by specifying how the processing unit transitions between states, thereby transforming the transistors or other discrete hardware elements constituting the processing unit.

Encoding the program modules may also transform the physical structure of the computer-storage media. The specific transformation of physical structure may depend on various factors, in different implementations of this description. Examples of such factors may include but are not limited to the technology used to implement the computer-storage media, whether the computer storage media are characterized as primary or secondary storage, and the like. For example, if the computer-storage media are implemented as semiconductor-based memory, the program modules may transform the physical state of the semiconductor memory, when the software is encoded therein. For example, the program modules may transform the state of transistors, capacitors, or other discrete circuit elements constituting the semiconductor memory.

As another example, the computer-storage media may be implemented using magnetic or optical technology. In such implementations, the program modules may transform the physical state of magnetic or optical media, when the software is encoded therein. These transformations may include altering the magnetic characteristics of particular locations within given magnetic media. These transformations may also include altering the physical features or characteristics of particular locations within given optical media, to change the optical characteristics of those locations. Other transformations of physical media are possible without departing from the scope of the present description, with the foregoing examples provided only to facilitate this discussion.

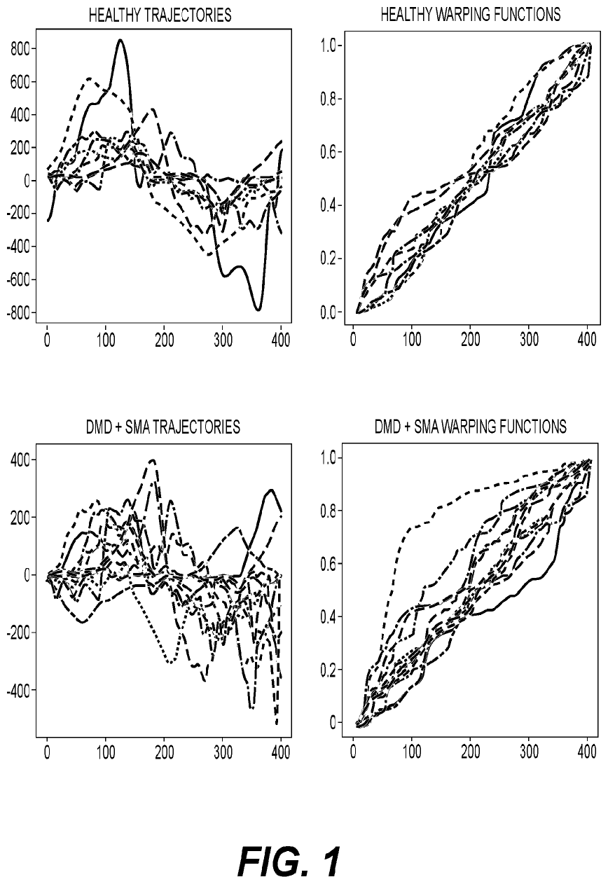

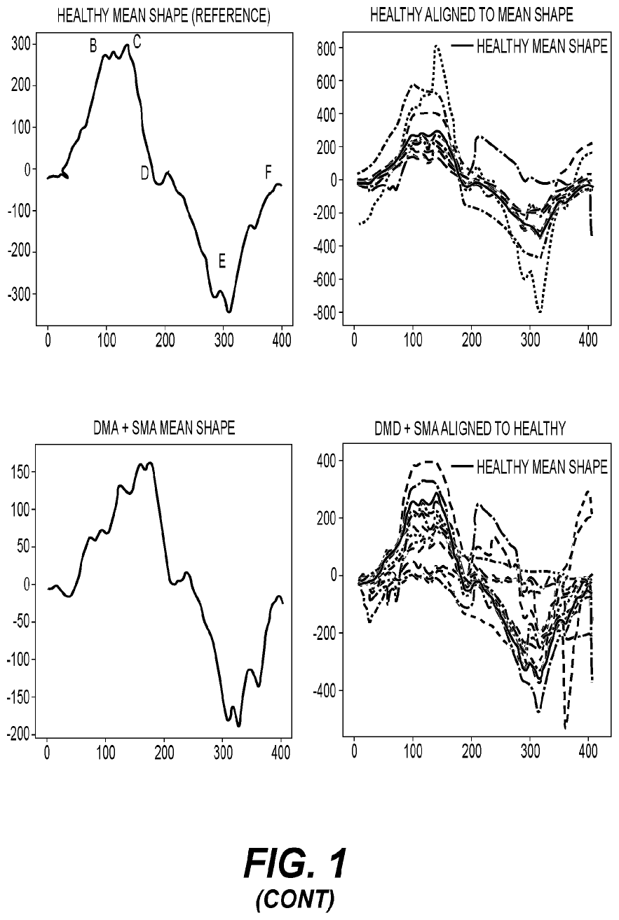

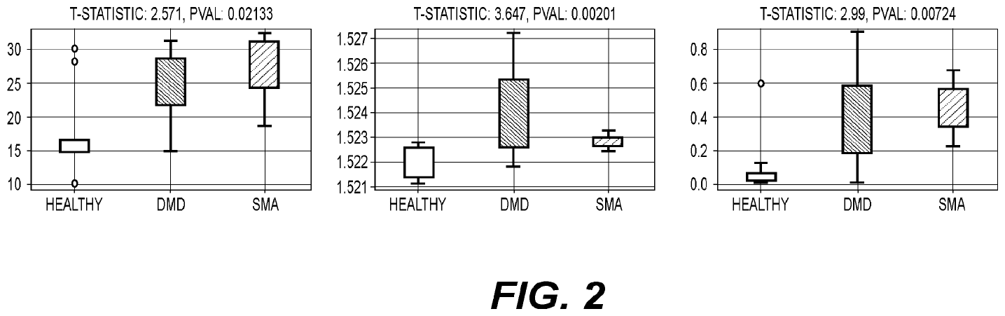

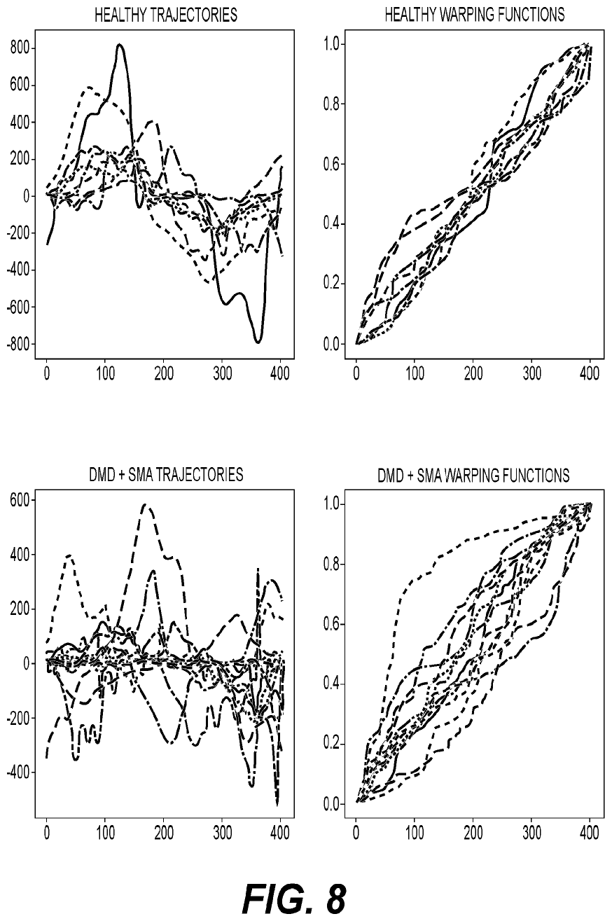

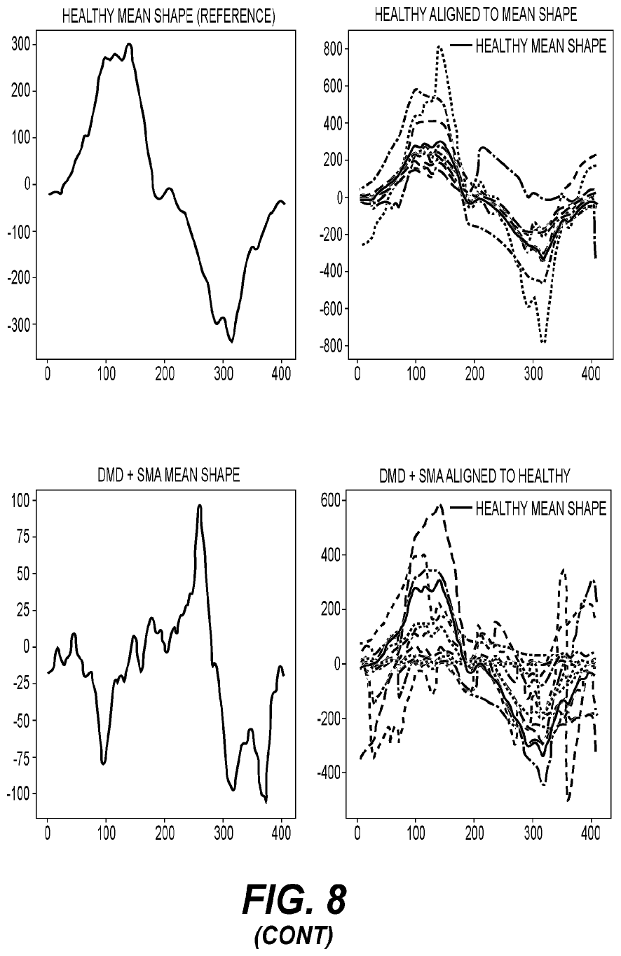

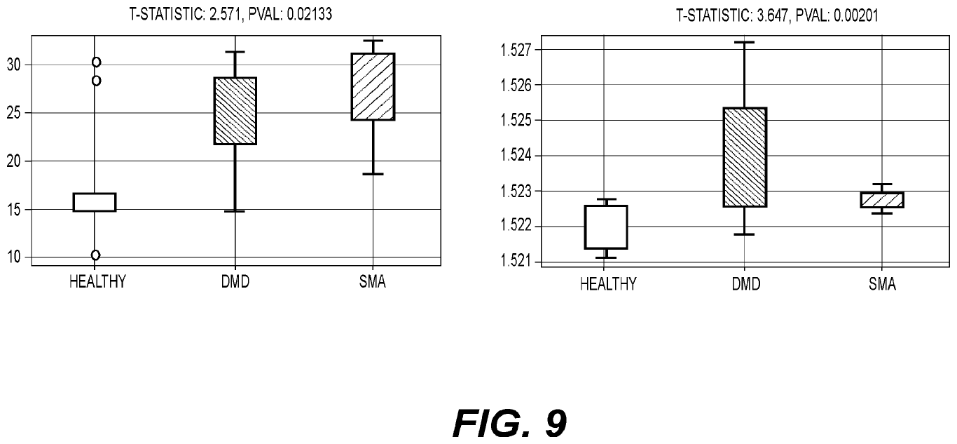

This paper uses curve registration and shape analysis that is graphically illustrated in FIG. 1 and FIG. 8 to temporally align trajectories while simultaneously extracting a mean reference shape. Distances from this mean shape are used to assess the quality of motion. The proposed metric is invariant to confounding factors, such as phase variability, while suggesting several clinically relevant insights. As shown in FIGS. 2-4 and FIG. 9, there are statistically significant differences between functional scores for the control and patient populations (p=0.0213≤0.05). Next, several patients in the patient cohort are able to perform motion on par with the healthy cohort and vice versa. One new metric, which is computed based on wearables, is related to the Brooke's score ((p=0.00063≤0.05)), as well as motor function assessments based on dynamometry ((p=0.0006≤0.05)). These results show promise towards ubiquitous motion quality assessment in daily life.

One non-limiting goal of this disclosure is to produce a novel, multi-modal platform capable of measuring fine-grained muscle structure and function in patients with neuromuscular diseases that will revolutionize the way clinical trials are performed moving forward, therefore accelerating the pipeline of new treatments for childhood neuromuscular diseases. This disclosure hypothesizes that the novel measures of upper extremity muscle structure and function proposed here will be able to distinguish small changes and differences in function that are not measurable by current clinical metrics. In following these patients, clinicians have identified multiple ways in which the motor function evaluation could be dramatically improved.

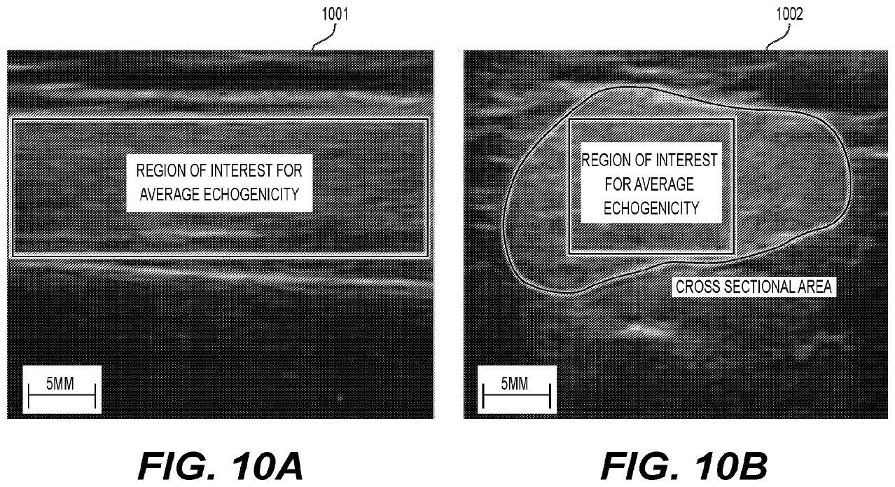

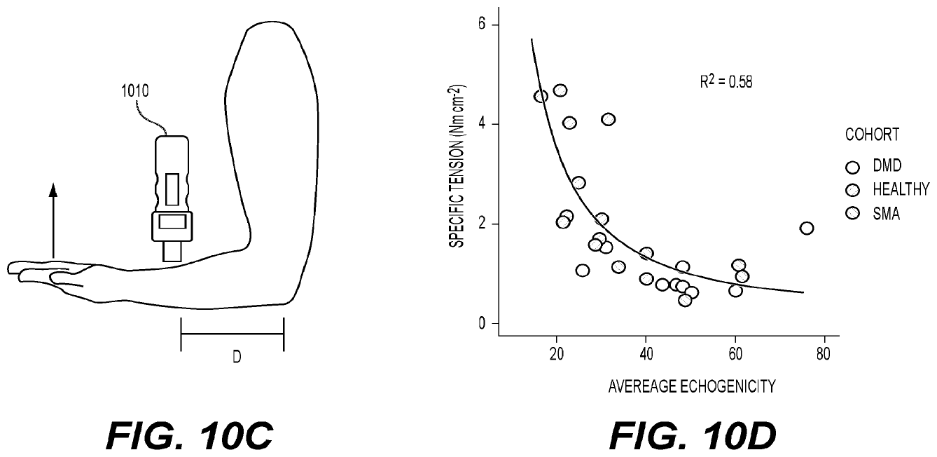

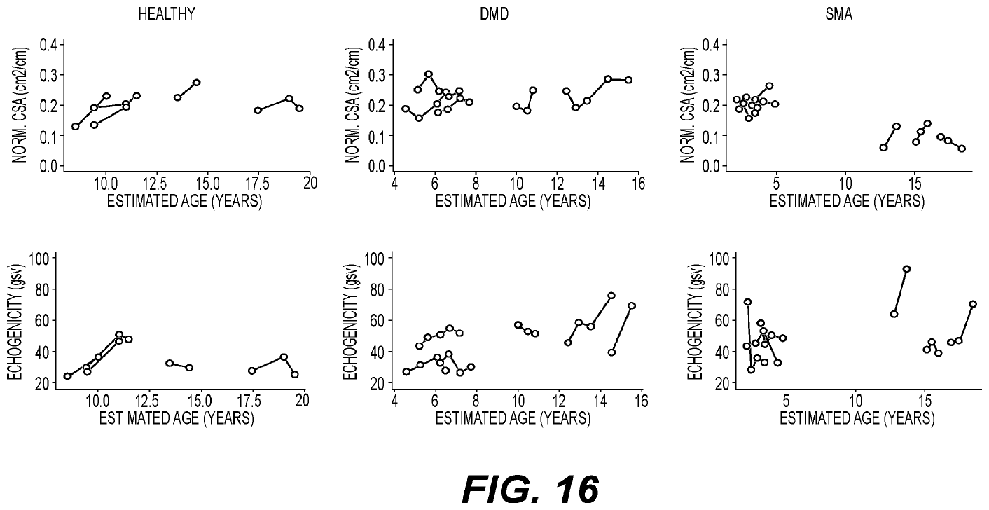

The traditional motor measurement, the CHOP-INTEND of FIG. 14, is an infant neuromuscular assessment; our patients range from age 2 to age 35 years. Therefore, the current assessment metrics are virtually irrelevant to most patients. Secondly, the metrics are performed in the clinic and therefore may have no bearing or relevance to the child's function in her/his normal environment. This has been frequently observed by parents who often report progress that is not captured by the standard clinical assessments. Clinical discoveries of this disclosure have found that while the patients may not be showing signs of progress to the CHOP INTEND, they have shown signs of motor function improvement. Finally, the current assessments are gross and qualitative and are not able to capture specific changes in muscle strength and function. By integrating multimodal motor assessment techniques, this disclosure hypothesizes that teams can develop a more objective scoring measure that could be eventually deployed for continuous, home-based monitoring. This disclosure shows that ultrasound measurements can also be an arbiter between the CHOP-INTEND and the wearable sensor measures enabling testing of the validity of the new measurement approach. See FIGS. 10A-10D, FIG. 15, and FIG. 16.

This disclosure shows developing and deploying the platform in a muscular dystrophy clinic, including the assessment of 29 patients and 15 age-matched controls. The results from pilot data illustrate the capability of the method to distinguish small but meaningful differences between patients in muscle structure and function to a degree that is not achievable by standard clinical measures. One of the non-limiting goals of this proposal is to extend trials to longitudinally track patients and examine whether the proposed methods can detect changes in muscle structure and function over long periods of time and outside of a clinic. Furthermore, others can use these findings to refine the platform such that it can be incorporated into a portable system that can be taken home by patients.

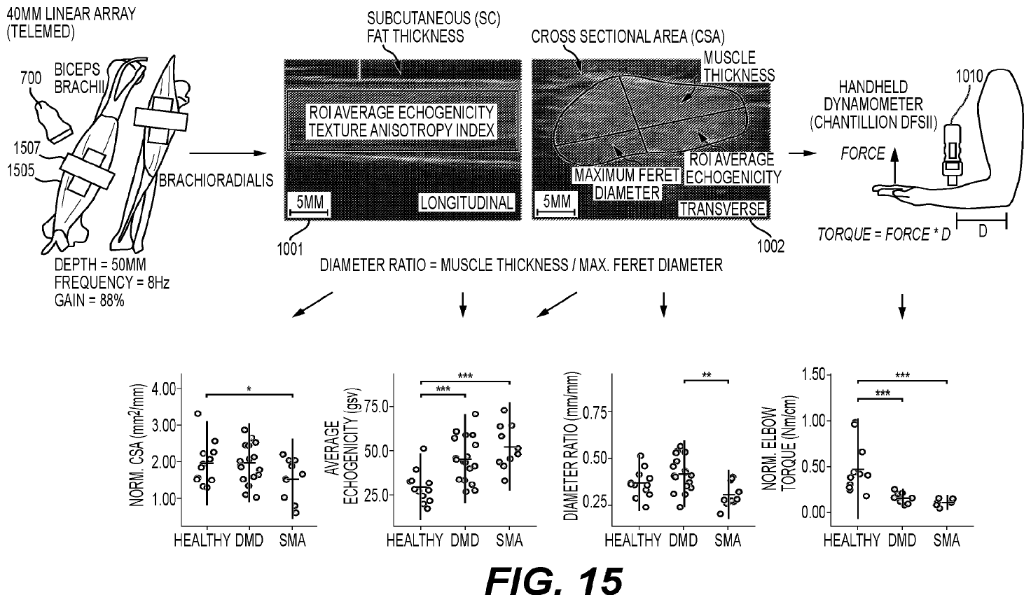

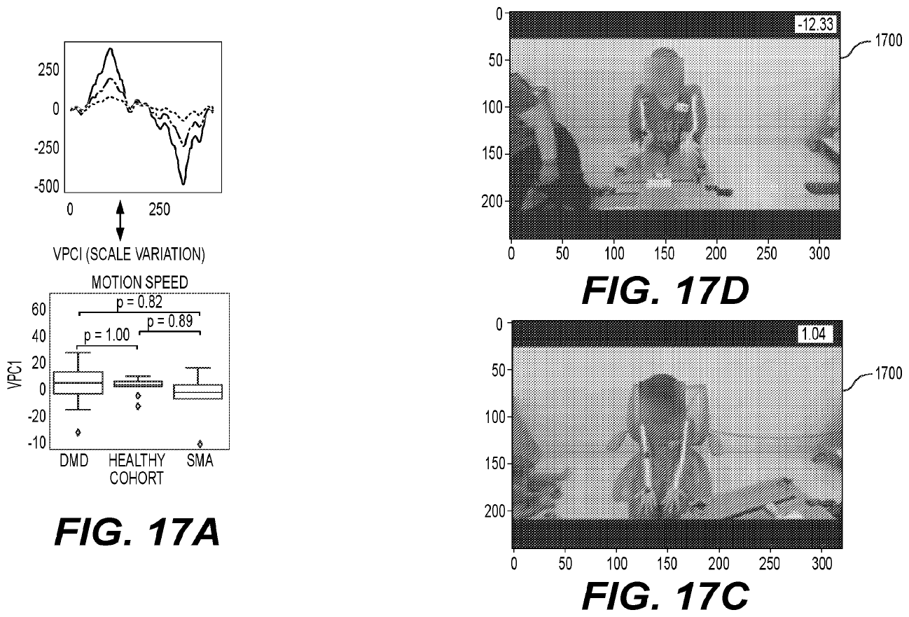

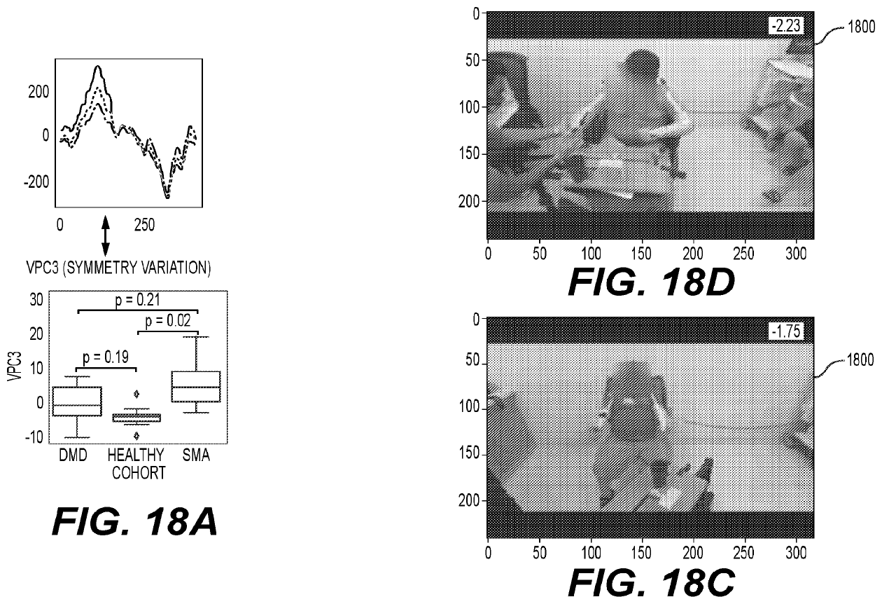

In one non-limiting embodiment, an imaging device 700 and wearable sensing system of FIGS. 17 and 18 are used to monitor changes in muscle structure and function longitudinally in a cohort of patients with neuromuscular disorders. The ultrasound 700 measurements will include quantification of key metrics of muscle architecture (anatomical cross-sectional area (ACSA), thickness, and tissue echogenicity) as shown in FIGS. 10A-10D and FIG. 15, specifically isolating the biceps and pronator muscles of the upper extremity. The ACSA and thickness measurements have been shown to predict the peak force generating potential of a muscle [9]. In pathological muscle, myotube development has been shown to be delayed therefore reducing over muscle function later in life [10]. Further, in diseased muscles, healthy muscle fibers are replaced by areas of fibrosis and fatty infiltration [11], [12]; therefore, the functionality of the muscle is reduced. The echogenicity of the tissue will reveal these areas and we will be able to effectively calculate a “functional” cross-sectional area to better relate to the muscle functionality tests. These metrics are important because, when developing treatments for pediatric neuromuscular disorders, it is imperative to measure the degree to which they allow for re-establishment of healthy muscle parameters.

The wearable sensing system, disclosed as one non-limiting embodiment, includes multimodal accelerometer and gyroscope sensors to continuously measure movements for assessing a full range of function within a clinic environment. The digital biomarkers extracted from these wireless sensors will measure movements from a child's wrist, hand, and arm to allow for tracking progress in patients with neuromuscular disorders receiving a new medication or physical therapy, including assessing activity and task performance that correlate with activities of daily living.

The aggregate data will aid in developing an objective, data-driven composite score, or system for assessing treatment outcomes to compare with the previously collected, insufficient metrics such as the CHOP-INTEND and HFMSE scores [1]. CHOP-INTEND is the current ‘gold standard’ for evaluating neuromuscular disorders on a CHOP-INTEND score sheet HFMSE is an accepted motor scale evaluation for people with SMA who are able to sit and walk. The Brooke Upper Extremity Scale is a 6-point scale used to classify upper extremity function for patients with DMD.) Based on cross-sectional data, a set of “function” and “muscle scores” are capable of distinguishing between patient groups. By fine tuning these aggregate scores, researchers will be able to assess their ability to detect small changes over time, as well as explore new metrics, including functional principal component analysis.

One non-limiting goal of this disclosure includes developing a minimalistic set of measures that can be deployed for continuous, home-based monitoring and objective clinical measures of muscle function. By assessing the composite score, research will be able to identify which functional and muscle score metrics are critical to assessing change over time. Based on this analysis, a modified system will be incorporated into a mobile device or a technology that could be taken home by the patients. Additionally, the ultrasound estimates of muscle function could be incorporated into the standard of care to supplement and add more objective data to the current clinical assessments of muscle function. This system and method will empower researchers to develop a larger, longitudinal clinical trial to fully test the technology in a large set of patients.

In non-limiting embodiments, ultrasound techniques can be used to study muscle structure, size and function, specifically isolating the biceps and pronator muscles of the upper extremity on the dominant arm with the muscle at rest. Post-processing the images in a custom MATLAB algorithm allows researchers to extract all measurements of interest and to calculate theoretical metrics of muscle functionality using established muscle mechanics relationships. To collect these measures, we will use a linear array probe to image the transverse plane 1505 and longitudinal plane 1507 of the mid-belly of the muscle. The image 1001, 1002 was then imported into the algorithm to be manually processed by overlaying lines and tracing regions of interest that will output the measurements of thickness and ACSA based on the scale of the image, similar to the commonly used software, (FIG. 10B). The region of interest (FIGS. 10A and 10B) is defined in both planes to determine the echogenicity using gray-scale analysis, then the two planar values are averaged. Multiple images, approximately five per plane, will be taken to ensure consistency of imaging technique. From these measurements this disclosure estimates the specific tension (force per unit area) and generate a muscle score that estimates the muscle functional capacity. The muscle score is calculated using image-based measurements of the ACSA divided by the average echogenicity of the muscle. Additionally, this disclosure collected dynamometry to calculate the maximum voluntary elbow flexor torque (FIG. 10C) as a uniform measure of muscle function to compare our estimates of muscle functional capacity.

Certain ultrasound outcomes include dominant arm brachioradialis and biceps muscle ACSA and tissue echogenicity. This disclosure shows that the estimated specific tension relates to the average echogenicity or quality of the muscle, demonstrating that a relationship between the ultrasound measurements to muscle functional capacity (FIG. 10D). Additionally, this disclosure includes comparing a muscle score to maximum voluntary elbow flexor torque to ensure the methods and systems are equipped to estimate the muscle functional capacity. In non-limiting embodiments, this disclosure makes measurements over three time periods of the study to longitudinally track structure and functional changes. Next steps include examining data for distributions and looking at trends in muscle measurements over time, and will examine the muscle ultrasound data by a variety of variables, including age, sex, time receiving treatment, therapies received, and type of neuromuscular disorder. These measures will identify early changes in muscle architecture that may ultimately be able to predict long-term improvement or deterioration in function.

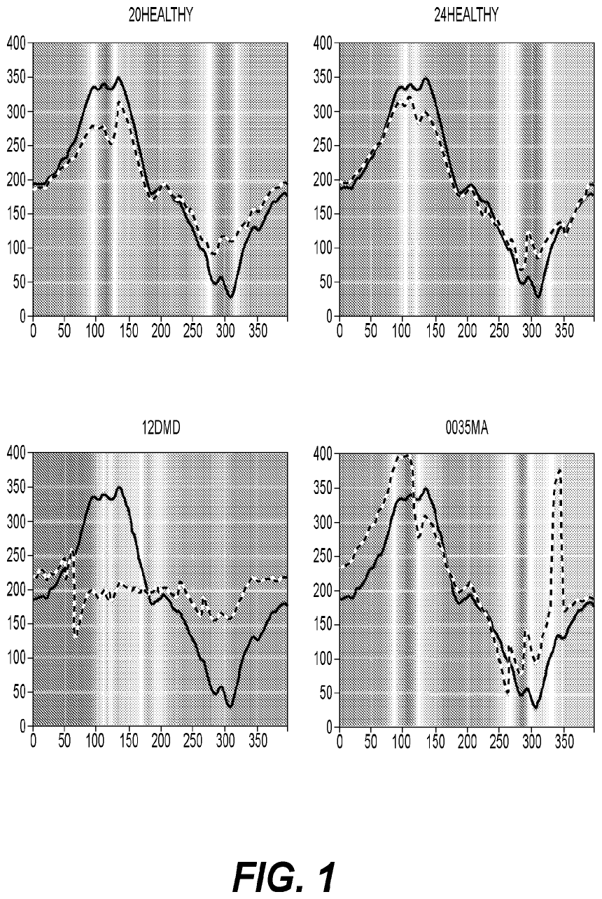

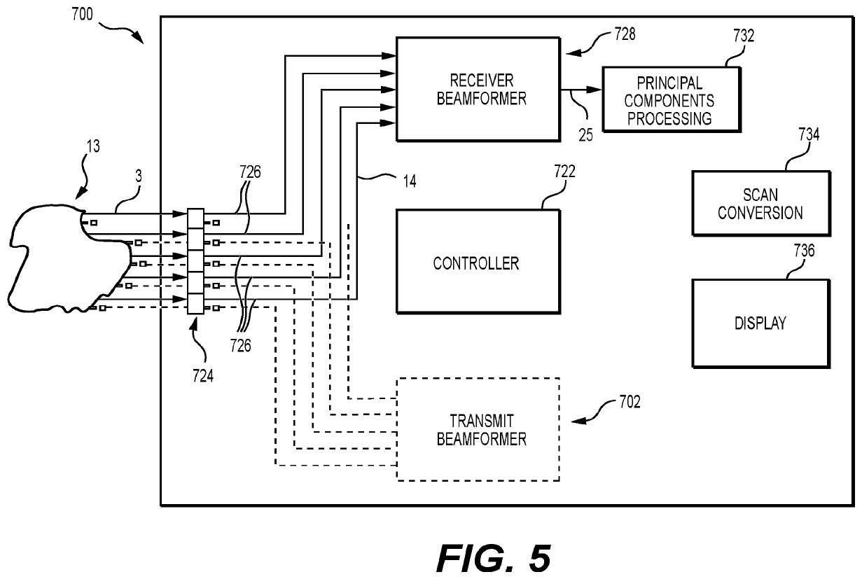

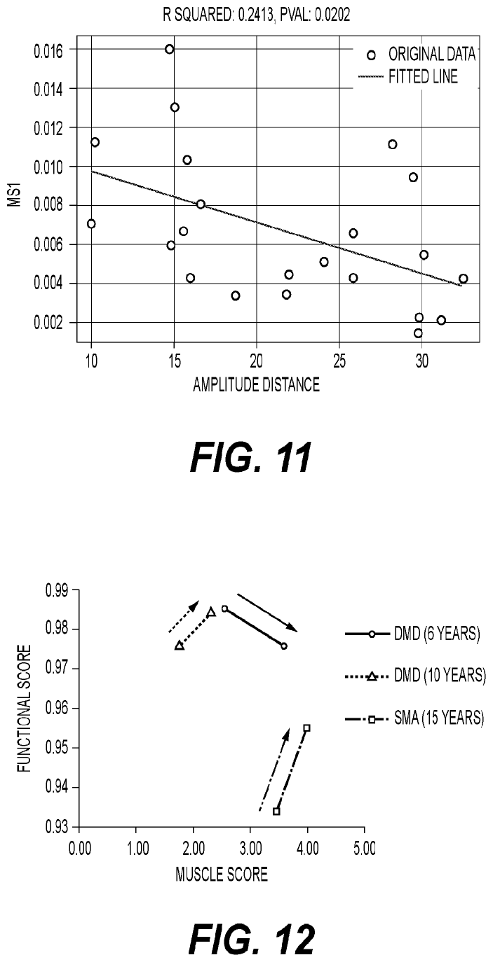

One non-limiting goal of this work is to create a composite index of FIG. 12 based on the functional and muscle score metrics that are critical to assessing change over time. To develop the composite index, this disclosures includes, but is not limited to, using the amplitude and phase distances from the healthy mean by performing phase amplitude separation [22]. The deviations computed from the mean shape of these aligned trajectories are used to quantify the quality of motion. Results show that functional movement scores based on shape analysis can capture quality of motion obtained from more fine-grained modalities like ultrasound. To do so, research compares the relationship of the indices (FIGS. 5 and 11): amplitude distance and phase distance with the muscle score obtained from ultrasound (plot below). The amplitude distance, in particular has a statistically significant linear relationships with the muscle score (which were captured from ultrasound). The preliminary results show promise towards using wearable technologies for continuous home-based monitoring of motion quality.

Collecting and leveraging longitudinal patient data, these metrics will be fine-tuned and aggregated to develop a composite score to compare data from the Brooke's score, which is the current test used in clinical trials and required by insurance companies to monitor progress in patients with severe neuromuscular disorders. This disclosure, in one non-limiting example, seeks to develop a composite score or system for determining outcomes that account for information that has never been captured. This study will provide concrete measures for researchers to identify the success, partial success, or inadequacy of research therapies, and devote resources accordingly.

An example of this phase variability can be seen in FIG. 1, first column. Here, several examples of arm curls are shown for both healthy and patient cohort. There is significant phase variability in the healthy cohort with peaks and valleys for different participants occurring at different times. This variability makes it challenging to compare and analyze these trajectories. Furthermore, to tempo-rally align these trajectories, we need to first establish a reference trajectory against which to align them. One good candidate is the mean of these trajectories; however one must first find a mean shape for the trajectories, which first requires temporal alignment to find the reference. In this work, the research demonstrates that this problem can be cast naturally in the language of differential geometry and shape analysis [1, 4, 7, 8]. First, by working with joint angles in test representations, the research avoids variations due to limb length, which is especially important considering participants have a wide variation in age and size. Then, the process includes phase amplitude separation, an iterative procedure to simultaneously discover a mean shape of the trajectories, while temporally aligning the trajectories with this mean shape. Next, this disclosure constructs three distances from this mean shape based on (i) amplitude, (ii) phase differences and (iii) cosine dissimilarity. In non-limiting examples, this research illustrates that by using the distances between aligned curves, clusters in the data become apparent which leads to more sensible classification. This also demonstrates that amplitude distances are related to motor function assessed from Brooke's score as well as from dynamometry. This is the first work using shape analysis for neuromuscular motion quality assessment.

### Methodology

Through the Pediatric Neuromuscular Clinic at the University of Virginia Children's Hospital, data was collected from 41 participants. Patients were either diagnosed with SMA or DMD and control participants were recruited based on the enrolled patients, matching both age and sex (±1 year). Researchers placed a MetaMotionR+ (MbientLab, San Francisco, CA, USA) sensor on the top of each hand, with the accelerometer and gyroscope sensors sampled at 200 Hz. Then the researchers had each subject perform a series of motions related to activities of daily living, such as turning a door knob, raising a cup to their mouth, etc. The Brooke Upper Extremity Scale was used to provide one standardized metric for comparison between all cohorts [2]. The study was approved by the University of Virginia's Internal Review Board for Health Sciences Research, protocol #12161. For this study, some patients' data has been excluded from the subsequent analysis for the following reasons: 1. sensor malfunction (2); 2. refusal to cooperate due to young age (2); 3. patient withdrew from study (1).

The methodology of calculating test patient amplitude distance from a mean amplitude of healthy patients as well as calculating test patient phase distance from a mean phase distance of healthy patients includes the following steps.
Let βt be the function we are interested in modeling. In our case, it represents the gyroscope's signal. Given raw data, we resample, smooth, and find its derivative to first obtain β′(t). Our initial preprocessing step for reducing noise in the data included the use of a 3rd order, low-pass Butterworth filter with a cutoff frequency of 0.1 Hz.
The square root velocity function (srvf) ([8]) of a function is then defined as: qₜ=β′(t) divided by the Square Root of the Absolute Value of β′(t). This allows the research to define the following two distances:

$$A ( β1 ( t ) , β2 ( t ) ) = inf γ ‖ q 1 - q 2 ∘ γ √ γ ’ ‖$$

Similarly, after finding the optimal warping function which aligns two functions, the phase distance between them is given by:

$$P ( γ a \to b ) = cos - 1 ( < γ id , √ γ a \to b > ) .$$

In order to compute the pairwise distance matrices because some of the trajectories were particularly noisy, this disclosure also computes the cosine distance between the aligned functions:

$$C ( β1 ( t ) , β2 ( t ) ) = 1. - {cos}^{- 1} ( < β1 ( t ) , β t ( t ) > )$$

Once the srvf has been defined, one can perform phase amplitude separation as outlined in to align the trajectories and also calculate the mean shape. Non-limiting examples use the python package f dasrs f for our analysis.

In FIG. 1, this disclosure shows the results of performing phase amplitude separation on a dataset. The first group comprises a set of healthy trajectories, which is shown in the top left plot. The second group comprises of DMD+SMA, whose trajectories are shown below. As one can see from the raw trajectories and the healthy cohort in particular, the data has a lot of phase variability, y meaning that despite having the similar shapes, the peaks and valleys occur at different times in different trajectories. The next step is to run phase-amplitude separation on the set of healthy trajectories, as described in the section above. This aligns these functions temporally while simultaneously discovering a mean shape. The elastic mean shape of healthy arm curls is shown in the third plot, while the corresponding temporal warping functions are shown in the second plot.

The mean shape has a rather interesting structure. It consists of a stationary phase when the curl is initiated (A). The peak angular velocity is not reached immediately but through transient stage (B) which involves a slight deceleration after which peak velocity is reached (C). From looking at the videos, we discovered that the twin peaks (B to C) is because of a slight flicking of the wrist toward the shoulder. What is even more interesting is that the mean shape does not smoothly decelerate to 0, first overshooting it a little (D) and then coming back to 0. Similarly, in the deceleration phase, involves two peaks (E) followed by return to 0 (F). The symmetrical shape of the motion indicates that the participant performed the same amount of rotation up and down. The warping functions provide a sense of how much work needs to be done in order to map each function to the healthy mean. From the top right plot, one can see that the peaks and valleys of the healthy trajectories align quite nicely with the mean shape.

In the second row, the corresponding DMD+SMA trajectories are aligned temporally to the elastic healthy mean. The first plot shows the raw data while the second plot shows the warping needed in order to align the unhealthy trajectories with healthy. In the rightmost plot, this disclosure overlays the aligned trajectories on the elastic mean. Here one sees much poorer agreement between the peaks and valleys of the DMD+SMA cohort and the elastic mean. As one can see visually, the warping functions for the DMD+SMA cohorts are a lot more distorted, implying that the DMD+SMA cohorts need to be “stretched and squished” a lot more in order to align them to the healthy mean compared to the warping needed for healthy trajectories. This disclosure calculates the elastic mean corresponding to the DMD+SMA cohort. Because of the large variation in trajectories, this elastic mean is much more noisier.

This disclosure computes the elastic phase and amplitude distance to the healthy mean. The distribution of the aforementioned distances is shown in FIG. 2. As one can see, the DMD+SMA cohorts have larger amplitude, phase and cosine distances compared to healthy mean. Researchers run a t-test with unequal variance assumption to compare DMD+SMA with healthy. For amplitude distances, the test statistic is 2.571 and the p-value is 0.02133. For phase distances, the test statistic is 3.647, while the p value is 0.00201 Finally, for the cosine distances, the test statistic is 2.99 with a p value of 0.00724. Thus amplitude, phase and cosine distances from the registered mean seem to capture differences between the healthy and non-healthy cohorts. In all three cases, the healthy cohort had much lesser variance than DMD. Another subtlety that the research showed was that the amplitude distance had an extra outlier (participant 17) which was not present in the phase and cosine similarity distances. This is because participant 17 had performed the motion almost 3 times as fast as the other ones. The amplitude distance being based in frobenius norm is more sensitive to larger magnitudes while the cosine dissimilarity (and also the phase distance) because of its normalization term is less susceptible to such larger velocities.

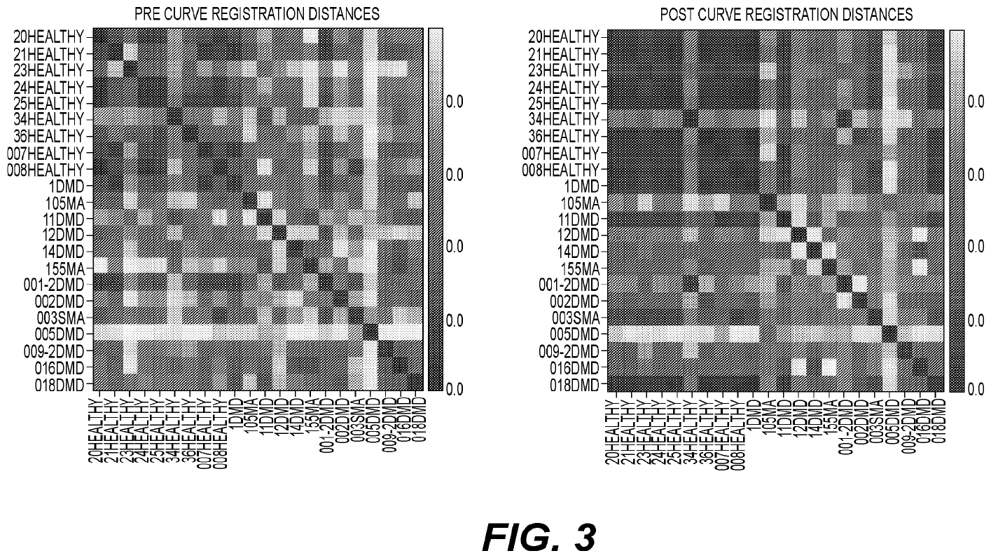

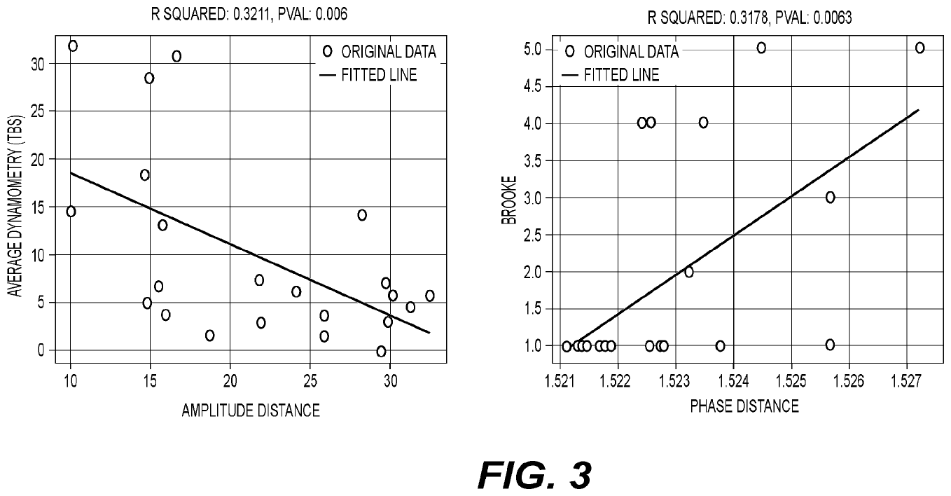

In order to establish the utility of curve registration, In FIG. 3 the research plots pairwise cosine distances between points pre and post curve registration. This disclosure used cosine distance instead of the elastic distance as some of the DMD+SMA trajectories are really noisy and elastic distance involves an alignment step which performs poorly with the noisy trajectories. The participants have been ordered by cohort with all the healthy ones being close to each other. As one can see, the distance matrix on the left does not have a visually discernible block structure of any sort, which would indicate clustering. This is not surprising since the presence of phase variability in data obfuscates some of the structure present. The distance matrix post curve registration seems to have a clear block structure with the set of healthy controls having much smaller distances from each other. Interestingly, some of the participants with DMD (18 and 1) seem to have smaller distances than the healthy participants despite belonging to the DMD class. The healthy participants (34) seem to have much larger distances than the non-healthy cohorts.

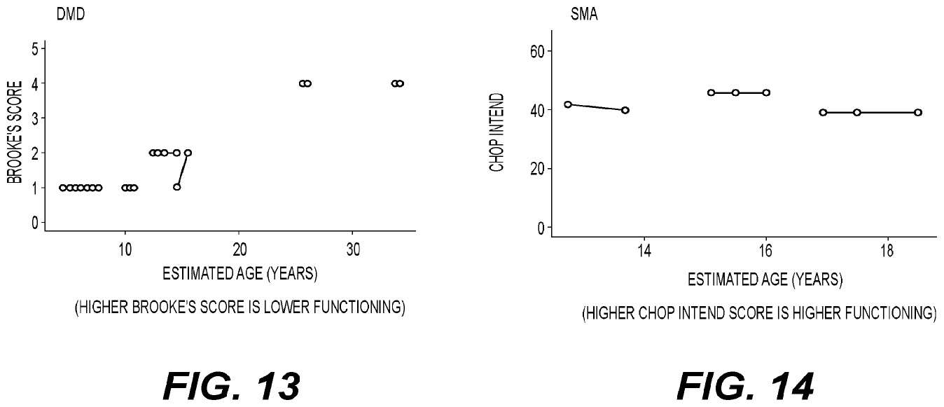

One non-limiting aim of this work is to develop a composite index of symptom severity based on unsupervised motion data. To do so, non-limiting examples compare the relationship of certain predictors, including amplitude distance and phase distance with the Brookes score as well as the patient strength measured from dynamometry (FIG. 3) The amplitude distance had a statistically significant linear relationship with the dynamometry index (p=0.006≤0.05). This makes sense because patients with larger muscle strength were able to better perform the motion and have a lower amplitude distance. The phase distance has a statistically significant linear relationships with the Brooke's score of FIG. 13 which is the current clinical gold standard for assessing motor function ((p=0.0337≤0.05)). Larger phase distances imply larger Brookes score, indicating greater symptom severity. These preliminary results show promise towards using wearable technologies to capture movement patterns and daily life and augment time-consuming, less frequent physiological assessments requiring expensive lab grade equipment and subjective measures like the CHOP-INTEND.

The long-term goal of this work is to develop an index for assessing motion quality over time. This disclosure identifies salient patterns across healthy controls, DMD, and SMA participants. In FIG. 4, the team plots the rolling correlation between the reference aligned signal with the healthy mean. Participants 20 and 24 from the healthy cohort perform the arm curl motion very similar to the elastic mean, with differences at the peak and valley. The difference at the peak translates to how much of a wrist flick occurred when reaching the shoulder, while the valley is indicative of whether they returned their arm to the original starting position, or continued downward to their leg. Participant 12 with DMD on the other hand, seems to not be able to perform the first part of the motion at all, which was expected due to their limited muscle strength. Participant 3 with SMA does a good job in the first part of the curl, while struggling with the last part of the curl, as they had difficulty rotating back to the original starting position.

This work presents evidence for how wearables coupled with shape analysis can allow us to come up with a metric for assessing motion quality. Phase amplitude separation allowed researchers to overcome issues with phase variability and capture functional differences between the healthy and patient cohorts. Moreover, a new metric was able to detect subtle differences for some of the non-healthy participants that would have otherwise gone unnoticed. The rolling correlation between registered trajectories allowed us to understand when parts of the motion were performed well and when it deviated from the accepted path. This research shows a statistically significant relationship between functional muscle scores (i.e., functional movement scores) and the muscle score obtained from ultrasound and dynamometry.

Results here have several important implications: One can imagine using such a system for home use, where one can track data from patients with neuromuscular disorders over longer durations and without burdening the patient to come into the clinic multiple times. This will not only allow doctors to collect more data, but also aid doctors in tracking progress that is often noted from patient caregivers. By coupling such a system with activity recognition classifiers, it's possible for a clinician to monitor function progress in a variety everyday activities. Finally, the non-intrusive nature of the monitoring system makes it possible to track longitudinal progression, which is especially important for patients who have received revolutionary treatments like gene therapy. Telemedicine with wearable sensors will allow clinicians to offer support to pa-tients during events like pandemics and more remote areas of the world.

### Examples

Practice of an aspect of an embodiment (or embodiments) of the invention will be still more fully understood from the following examples and experimental results, which are presented herein for illustration only and should not be construed as limiting the invention in any way.

Upper EXTremity Examination for Neuromuscular Diseases (U-EXTEND): Objectively Assessing Treatment Efficacy and Accelerating Drug Discovery

With emerging therapies in neuromuscular disorders, monitoring progress is vital. For example, Spinal Muscular Atrophy (SMA) is the leading genetic cause of death in infants with a life expectancy of less than 2 years, which until recently, had no treatment for this disease. However, the development of Nusinersen and other forms of treatment have led to a historic moment in the care of patients with SMA which cannot be evaluated with current assessment tools. Similarly, new treatments are being developed for Duchenne Muscular Dystrophy (DMD), the most common muscular dystrophy, and the most common genetic cause of death in boys. These treatments have been slow to progress through clinical trials and to reach patients, in part due to lack of sensitive and specific markers of improvement which can be used to monitor effectiveness. As with any medical treatment, the outcomes vary from patient to patient. Therefore, the present disclosure submits that its new research must be done to measure the effectiveness of interventions (or lack thereof) and motor change-over in neuromuscular disorders, from micro-movements to muscular regeneration, in a group of patients who previously did not live long enough to keep measures of progress. An aspect of an embodiment of the present system and method will produce a novel, multi-modal platform of measuring motor function in patients with neuromuscular diseases that will revolutionize the way clinical trials are performed moving forward, therefore accelerating the pipeline of new treatments for childhood neuromuscular diseases. The present disclosures hypothesizes that the novel measures of upper extremity muscle structure and function proposed here will be able to distinguish small changes and differences in function that are not measurable by current clinical metrics.

Translational applications-Clinical situations of this disclosure include careful and accurate measure of arm muscle function would promote care for patients with neuromuscular disorders: 1) Using reliable and quantifiable evaluation results, we can determine what treatments are, or are not, supporting progression or sustainment of muscle function, do this more quickly, and align effective therapies appropriately. 2) By developing and sharing these evaluation tools and methodologies, clinical researchers will be able to measure the efficacy of SMA therapies in investigation, adjust focus and investments to promising developments, and mitigate investments on non-performing studies, thereby speeding discovery. In addition, research in these pioneer areas of severe muscular diseases could become more feasible or compelling with the existence of meaningful, reliable, quantifiable measuring tools. 3) This paradigm could also be applied for use in Duchenne Muscular Dystrophy (DMD), Cerebral Palsy (CP), Amyotrophic Lateral Sclerosis (ALS) and other neuromuscular disease patients in severe or end stages of disease, where fine motor skills of the hands and fingers may be the remaining ability. 4) The wireless sensing technology calibrated and adapted to enable measurement of muscle movement and function in muscle sets and ranges meaningful to other neuromuscular disease patient populations. This approach also has the potential of being used in a natural living environment during a variety of activities of daily living (ADLs) or even could be integrated into a video game or tablet application.

### Data Gathering Techniques

This disclosure has piloted the use of our combined ultrasound and wearable sensing system in a set of 29 patients, including 10 with SMA, 19 with DMD, and 15 age-matched typically developing patients. Additionally, researchers have collected longitudinal ultrasound and motion data for 5 SMA and 7 DMD patients resulting in a very rich data set for this small pediatric population. In order to transform all these data into meaningful clinical measures, we have created a scoring system for wearable and ultrasound data.

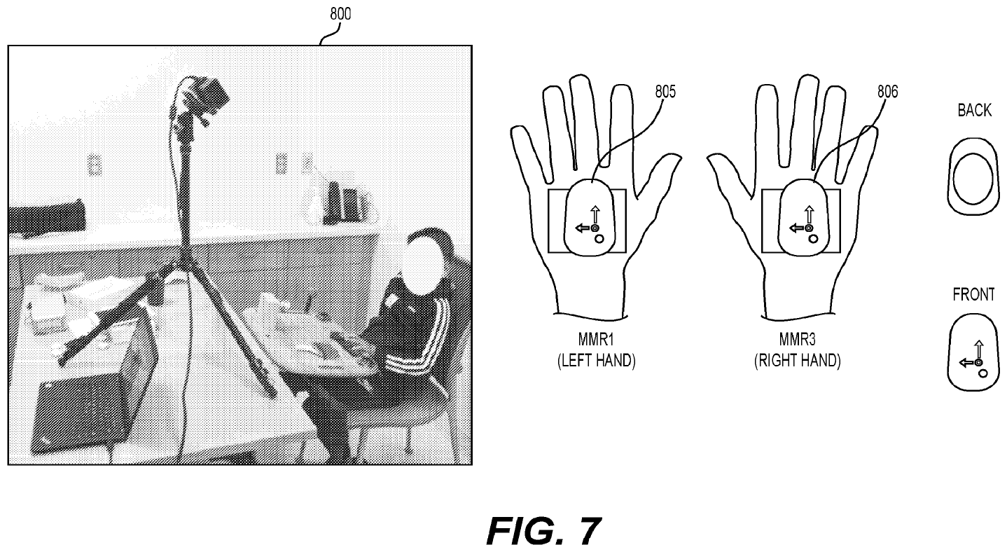

This research has designed a study utilizing multimodal wireless inertial sensors and video-based motion tracking to measure movements in space as shown in FIG. 7. Attaching sensor nodes to a patient's upper extremities (number of sensors and exact positioning will be determined in the lab) will allow us to measure progress with medication or during physical therapy in small gradations to detect micro-movements. Current research [13]-[15] has shown that video-based tracking can accurately measure natural movements and full range of function, for all patients with neuromuscular disorders, especially toddlers and babies. Non-limiting examples herein will gather measurements from both dominant and nondominant arms, having the patient press against the manometer and pinch the grip strength gauge with each hand or arm. For each task, three measurements will be collected and the strongest for each will be used. Our collaborative team will do the same for the wireless sensor node, to be worn on both wrists. Through a series of prescribed motions, as described in the Revised Upper Limb Module (RULM) for SMA (e.g., pick up tokens, raise a cup), patients will perform tasks related to everyday activities [16], [17]. The wireless sensing technology will be first piloted in a patient who is part of the healthy control group to adjust and calibrate our protocol, and then will be used in the full cohort of 45 participants. These controlled data collections will be done in the clinic to drive the development and validation of sensing requirements (both sensors and sampling rates) and data analytics methods for motion assessment in this patient population, thereby enabling subsequent out of clinic deployments for continuous monitoring. Measures of grip strength, manometer measurements of forearm pronation and biceps curl, accelerometer/gyroscope measurements using Shimmer Sensing System (or similar), and functional reaching volume (FRV) will be collected while the patient performs prescribed motions.

This disclosure further used functional data analysis for analyzing pediatric motion quality. The first group in FIG. 2 comprises a set of healthy trajectories for a bicep curl, which is shown in the top left plot. The second group comprises DMD+SMA, whose trajectories are shown below. Since the patients can perform the arm curl motion at different speeds, we need to align the trajectories with each other and obtain a mean shape. After running phase amplitude separation on the set of healthy trajectories to align these functions temporally, we show the elastic mean shape of healthy arm curl in the third plot while the corresponding temporal warping functions are shown in the second plot. The warping functions provide a sense of how much distortion needs to be done in order to map each function to the healthy mean. In the second row, the corresponding DMD+SMA trajectories are aligned to the elastic healthy trajectory. This disclosure also calculates the elastic mean corresponding to the DMD+SMA cohort. Because of the large variation in trajectories, this elastic mean looks less structured. Plots show the warping needed in order to align the unhealthy trajectories with healthy seems much larger than the warping needed for healthy trajectories.

In FIG. 3, this disclosure computes the elastic phase and amplitude distance to the healthy mean. The distribution of the aforementioned distances is shown in FIG. 2. As one can see, the DMD+SMA cohorts have larger amplitude and phase distances compared to healthy ones. Researchers run a t-test with unequal variance assumption to compare DMD+SMA with healthy controls. For amplitude distances, the test statistic is 2.571 and the p value is 0.02133, which is statistically significant. For phase distances, the test statistic is 3.647 while the p value is 0.00201 which is again statistically significant. Thus, both amplitude and phase distances from the mean seem to capture severity. Research uses the amplitude and phase distances from the healthy mean shape as a functional score to capture motion quality.

Additional descriptions of aspects of the present disclosure will now be provided with reference to the accompanying drawings. The drawings form a part hereof and show, by way of illustration, specific embodiments or examples. It should be appreciated that a variety of ultrasound related systems and methods (and computer readable medium) may be utilized as part of implementing or practicing aspects of the various embodiments of the present invention.

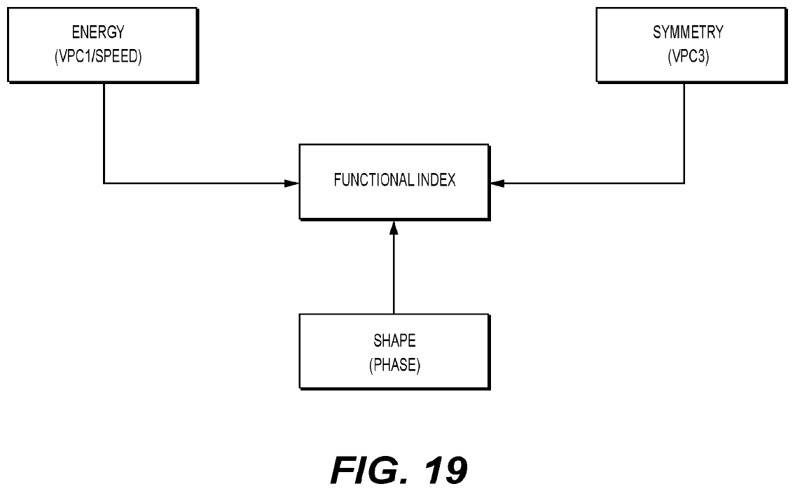

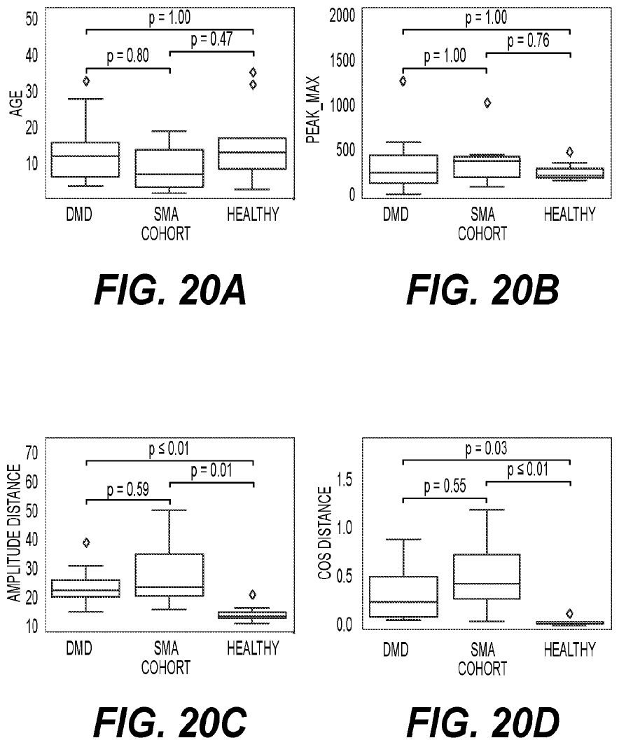

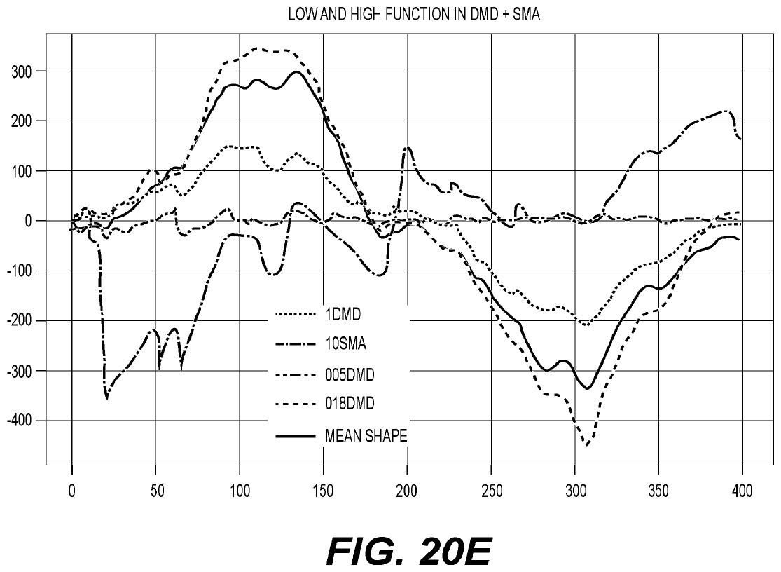

In an implementation, a computerized system calculates a respective composite index for respective sets of functional movement scores that quantify motor function abilities for patients diagnosed with a disease. A wearable sensor 805, 806 of FIG. 7 is positioned proximately to a test patient's anatomy to gather test motion shape data and test motion symmetry data. A respective wearable sensor is positioned proximately to a control patient's anatomy to gather control motion shape data and control motion symmetry data. A kinetic energy sensor 1700, 1800 of FIGS. 17A-17C and 18A-18C is positioned proximately to a test patient's anatomy to gather test kinetic energy data. A respective control kinetic energy sensor positioned proximately to a control patient's anatomy to gather control kinetic energy data. A computer stores software implemented by a computer processor in communication with computer memory, wherein the computer has access to the test motion shape data, the test motion symmetry data, the control motion shape data, the control motion symmetry data, the test kinetic energy data, the control kinetic energy data, and wherein the software executes computerized steps with the computer processor to calculate and store the composite index from predictors of the functional movement scores. The predictors shown in FIG. 19 include a respective shape of motion factor, a respective motion symmetry factor, and a respective motion speed factor for each of the sets of functional movement scores. The computerized steps include calculating the respective shape of motion factor for each of the test motion shape data and the control motion shape data; calculating the respective motion symmetry factor for each of the test motion symmetry data and the control motion symmetry data; calculating the respective motion speed factor for each of the test kinetic energy data and the control kinetic energy data; and using the software to tabulate the respective composite index by calculating a probability that the respective shape of motion factor, the respective motion symmetry factor, and the respective motion speed factor correspond to one of the sets of functional movement scores.

FIGS. 17A-17C illustrate modes of variation representing kinetic energy of motion, performing the motion slower or faster. Positive value of VPC1 means reduction in angular speed. Here there are two examples of participants performing the motion slower (positive activation of VPC1, similar to yellow) and faster (negative activation of VPC1, similar to black).

Surprisingly, not much different between cohorts.

FIGS. 18A-18C show the second mode of variation that seems to capture asymmetry in the motion. Positive value of VPC3 means reduction in symmetry of motion. Here are two examples of participants with high activation of VPC3 (motion asymmetry dimension). We notice several participants struggling with top part of the motion (going against gravity) as opposed to bottom part of motion (more freefall).

Interestingly, patients with SMA seem to have higher asymmetry as opposed to DMD and SMA.

This disclosure identifies three important dimensions: Energy of motion, Shape of motion and Symmetry of motion. In order to work towards a joint wearable index, In terms of next steps, one can combine these dimensions (for instance via regression based approaches like proportional hazard models or machine learning approaches) to come up with a composite index. Having larger dataset can allow more robust evaluation of these models.

Curve Registration as discussed above is used to align the trajectories and derive two distances: amplitude (indicating deviation in angular velocity from reference trajectory) and cosine distance (indicating shape similarity) of FIG. 19. While the angular speed across cohorts tend to be similar, the distances between cohorts tend to be different with SMA showing more severe impairment in both amplitude and phase than DMD, which in turn is more impaired than healthy. DMD and SMA tend to have higher variance than Healthy and there are patients with high and low function within DMD itself. For instance, Patients 1 and 18 (green) have motor function almost on par with healthy. Compare these with 5 and 10 (red) which demonstrate significant impairment.

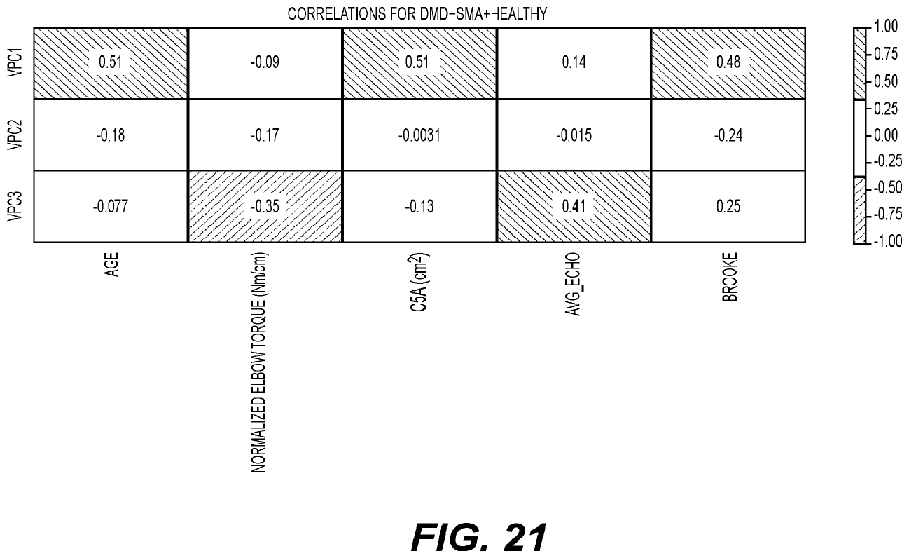

Considering FIGS. 17 and 18 and 21, this disclosure considers cross correlation between wearable features with clinical metrics. VPC1 (loss of angular speed dimension) is correlated with Age and dimensions correlated with Age (like Cross Sectional Area). The positive correlation implies that with increasing age, VPC1 increases (implying loss of angular speed). This is possibly because of the temporal degenerative nature of the disease.

VPC3 (loss of symmetry dimension) has lesser correlations with Age, negative correlation with dimensions like Normalized Elbow Torque (loss of symmetry implies loss of strength) and positive correlations with Echogenicity (loss of symmetry implies an increase in fat infiltration into the tissue).

In another implementation, calculating the probability includes assigning weights to the predictors and running the predictors through a regression analysis using machine learning software.

In another implementation, the system further includes an imaging device producing images of muscles of the test patient during motion assessment exercises and a dynamometer gathering measurements of force exerted by the muscles during the motion assessment exercise, wherein the computer receives the images and the dynamometer measurements and classifies corresponding muscle scores for the muscles, wherein the software uses the corresponding muscle scores as an additional predictor in tabulating the composite index.

In another implementation, the imaging device includes a linear array ultrasound probe that images a transverse plane and a longitudinal plane of the muscles.

In another implementation, the images include muscle measurements having anatomical cross sectional area (ACSA), muscle thickness, and tissue echogenicity.

In another implementation, the computer classifies corresponding muscle scores by calculating an anatomical cross sectional area (ACSA) of the muscles and dividing the ACSA by an average echogenicity of the muscle.

In another implementation, the wearable sensor and the respective wearable sensor each comprise a multimodal accelerometer.

In another implementation, the wearable sensor and the respective wearable sensor each further include e a gyroscope.

In another implementation a wearable sensor 805, 806 and the respective wearable sensor each include a wireless sensor in electronic communication with the computer.

In another implementation, the kinetic energy sensor 1700, 1800 and the respective control kinetic energy sensor each include a manometer measuring forearm pronation and bicep curl motion.

In another implementation, a video camera records video and audio data of the test patient during motion assessment exercises.

In another implementation, a computer implemented method assesses body movements performed by a test patient diagnosed with a disease, and the method includes gathering test motion data for the test patient with a sensor positioned proximately to the test patient's anatomy. The method further includes gathering control motion data for a control patient with a respective sensor positioned proximately to the control patient's anatomy and storing, in computer memory connected to a computer processor, test motion data trajectories and control motion data trajectories for respective sets of the test motion data and the control motion data. The method also includes using software accessible by the computer processor and the computer memory to perform computer automated steps including aligning the control motion data trajectories in the time domain; calculating a mean motion data trajectory for the control motion data trajectories; aligning the test motion data trajectories to the mean motion data trajectory in the time domain; and computing distance measurements between the test motion data trajectories and the mean motion data trajectory to quantify severity of motor function symptoms of the disease in the test patient.

In another implementation, computing distances includes computing at least one of an amplitude distance, a phase distance, or a cosine distance between the test motion data trajectories and the mean motion data trajectories.

In another implementation, computing distances includes computing a phase distance by computing a test motion data warping function to align the test motion data trajectories to the mean motion data trajectory.

In another implementation, the method includes, prior to aligning the control motion data trajectories, calculating the square root velocity function of the test motion data trajectories and the control motion data trajectories.

In another implementation, the method includes computing the distance measurements in the time domain across a range of values for the square root velocity functions of the test motion data trajectories to the mean motion data trajectory.

In another implementation, the computer implemented method of claim 12, further comprising using the software to complete phase amplitude separation on the test motion data trajectories and the control motion trajectories prior to calculating the mean motion data trajectory.

In another implementation, gathering the test motion data and the control motion data includes attaching the sensor to the test patient's anatomy and attaching the respective sensor to the control patient's anatomy.

In another implementation gathering the test motion data and the control motion data includes gathering data with a camera.

In another implementation, the method includes gathering muscle structure image data from the test patient and correlating the muscle structure image data with the distance measurements in the time domain.

In another implementation, the method includes gathering muscle structure data with an ultrasound image.

In another implementation, the method includes gathering the test motion data and the control motion data by wearing the sensor or the respective sensor on the body of a test patient or a control patient.

In another implementation, a computerized system calculates a respective composite index for sets of functional movement scores that quantify motor function abilities for patients diagnosed with a disease. A wearable sensor is positioned proximately to a test patient's anatomy to gather test motion shape data and test motion symmetry data. A respective wearable sensor is positioned proximately to a control patient's anatomy to gather control motion shape data and control motion symmetry data. A kinetic energy sensor is positioned proximately to a test patient's anatomy to gather test kinetic energy data. A respective control kinetic energy sensor positioned proximately to a control patient's anatomy to gather control kinetic energy data. A computer includes software implemented by a computer processor in communication with computer memory, wherein the computer has access to the test motion shape data, the test motion symmetry data, the control motion shape data, the control motion symmetry data, the test kinetic energy data, the control kinetic energy data, and the software executes computerized steps with the computer processor to calculate and store the composite index from predictors of the functional movement scores. The predictors include a respective shape of motion factor, a respective motion symmetry factor, and a respective motion speed factor for each of the sets of functional movement scores. The software implements steps of calculating the respective shape of motion factor for each of the test motion shape data and the control motion shape data; calculating the respective motion symmetry factor for each of the test motion symmetry data and the control motion symmetry data; calculating the respective motion speed factor for each of the test kinetic energy data and the control kinetic energy data; and using the software to tabulate the respective composite index by calculating a probability that the respective shape of motion factor, the respective motion symmetry factor, and the respective motion speed factor correspond to one of the sets of functional movement scores, wherein the shape of motion factor quantifies a severity of motor function symptoms of the disease in the test patient, and wherein the shape of motion factor is calculated according to a computer implemented method.

The shape of the motion factor is calculated by assessing body movements performed by a test patient diagnosed with a disease, and the method includes gathering test motion data for the test patient with a sensor positioned proximately to the test patient's anatomy. The method further includes gathering control motion data for a control patient with a respective sensor positioned proximately to the control patient's anatomy and storing, in computer memory connected to a computer processor, test motion data trajectories and control motion data trajectories for respective sets of the test motion data and the control motion data. The method also includes using software accessible by the computer processor and the computer memory to perform computer automated steps including aligning the control motion data trajectories in the time domain; calculating a mean motion data trajectory for the control motion data trajectories; aligning the test motion data trajectories to the mean motion data trajectory in the time domain; and computing distance measurements between the test motion data trajectories and the mean motion data trajectory to quantify severity of motor function symptoms of the disease in the test patient.

In another implementation, an imaging device producing images of muscles of the test patient during motion assessment exercises; and a dynamometer gathers measurements of force exerted by the muscles during the motion assessment exercise, wherein the computer receives the images and the dynamometer measurements and classifies corresponding muscle scores for the muscles; and wherein the software uses the corresponding muscle scores as an additional predictor in tabulating the composite index.

In another implementation, gathering the test motion data and the control motion data includes wearing the sensor or the respective sensor on the body of a test patient or a control patient.

FIG. 5 is a basic, schematic representation of an ultrasound system 700 according to an aspect of an embodiment of the present invention that is referred to in order to generally describe the operations of an ultrasound system to produce an image of an object 13. System 700 may optionally include a transmit beamformer 702 which may include input thereto by controller 722 to send electrical instructions to array 724 as to the specifics of the ultrasonic waves to be emitted by array 724. Alternatively, system 700 may be a receive only system and the emitted waves may be directed to the object 13 from an external source.

In either case, echoes 3 reflected by the object 13 (and surrounding environment) are received by array 724 and converted to electrical (e.g., radio frequency (RF)) signals 726 that are input to receive beamformer 728. Controller 722 may be external of the beamformer 728, as shown, or integrated therewith. Controller 722 automatically and dynamically changes the distances at which scan lines are performed (when a transmit beamformer 702 is included) and automatically and dynamically controls the receive beamformer 728 to receive signal data for scan lines at predetermined distances. Distance/depth is typically calculated assuming a constant speed of sound in tissue (e.g., 1540 m/s or as desired or required) and then time of flight is recorded such that the Additional descriptions of aspects of the present disclosure will now be provided with reference to the accompanying drawings. The drawings form a part hereof and show, by way of illustration, specific embodiments or examples.

It should be appreciated that a variety of ultrasound related systems and methods (and computer readable medium) may be utilized as part of implementing or practicing aspects of the various embodiments of the present invention.

The assembled output may be input into a scan converter module 734. The image formed within the scan converter 734 is displayed on display 736. Although FIG. 5 has been described as an ultrasound system, it is noted that transducers 724 may alternatively be transducers for converting electrical energy to forms of energy other than ultrasound and vice versa, including, but not limited to radio waves (e.g., where system 700 is configured for RADAR), visible light, infrared, ultraviolet, and/or other forms of sonic energy waves, including, but not limited to SONAR, or some other arbitrary signal of arbitrary dimensions greater than one (such as, for example, a signal that is emitted by a target).

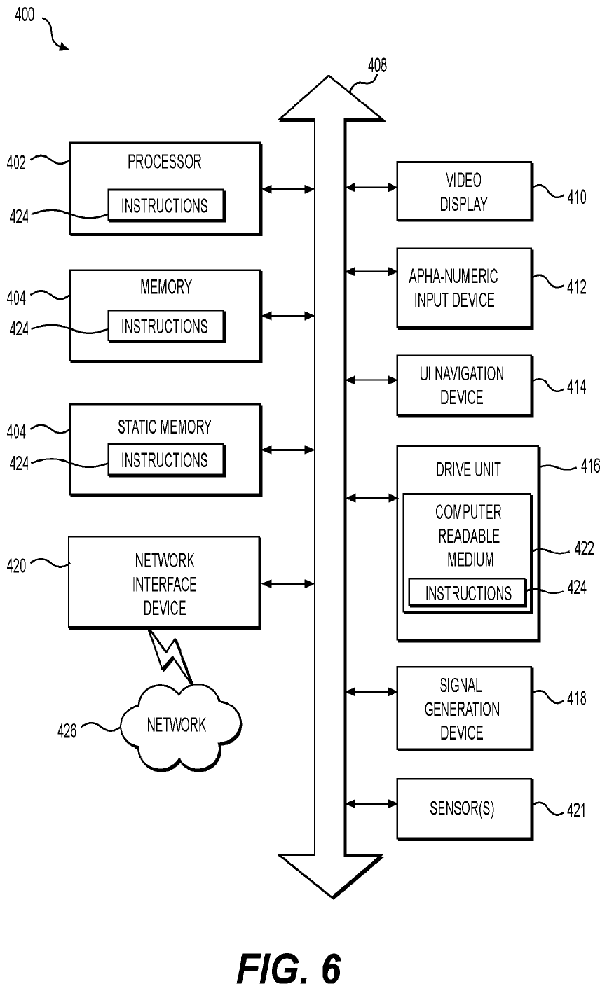

FIG. 6 is a block diagram illustrating an example of a machine upon which one or more aspects of embodiments of the present invention can be implemented. Referring to FIG. 6, an aspect of an embodiment of the present disclosure includes, but is not limited thereto, a system, method, and computer readable medium that provides one or more of any combination of the following: a) body motor function assessment; b) shape analysis for pediatric upper body motor function assessment; c) shape analysis for neuromuscular motion quality assessment; d) wearables in communication with shape analysis for generating a metric for assessing motion quality; e) track data from patients with neuromuscular disorders over longer durations and without burdening the patient; f) non-intrusive nature of the monitoring system that enables the tracking of longitudinal progression; g) multi-modal platform of measuring motor function in patients with neuromuscular diseases; h) measures of upper extremity muscle structure and function proposed here will be able to distinguish small changes and differences in function; i) multi-modal platform capable of measuring fine-grained muscle structure and function in patients with neuromuscular diseases; j) measures of upper extremity muscle structure and function proposed here will be able to distinguish small changes and differences in function that are not measurable by current clinical metrics; k) ultrasound techniques to study muscle structure, size and function, specifically isolating the biceps and pronator muscles of the upper extremity on the dominant arm with the muscle at rest; l) imaging and wearable sensing system to monitor changes in muscle structure and function longitudinally in a cohort of patients with neuromuscular disorders; m) based on analyses of the outcomes of the longitudinal data, develop a minimalistic set of measures that can be deployed for continuous, home-based monitoring and objective clinical measures of muscle function; n) wearable sensing system includes multimodal accelerometer and gyroscope sensors to continuously measure movements for assessing a full range of function within a clinic environment; and/or o) accelerometer and gyroscope sensors as a platform and would support home-based monitoring of upper extremity mobility which could enable more optimal and efficient longitudinal patient monitoring for clinical trials, which illustrates a block diagram of an example machine 400 upon which one or more embodiments (e.g., discussed methodologies) can be implemented (e.g., run).

Examples of machine 400 can include logic, one or more components, circuits (e.g., modules), or mechanisms. Circuits are tangible entities configured to perform certain operations. In an example, circuits can be arranged (e.g., internally or with respect to external entities such as other circuits) in a specified manner. In an example, one or more computer systems (e.g., a standalone, client or server computer system) or one or more hardware processors (processors) can be configured by software (e.g., instructions, an application portion, or an application) as a circuit that operates to perform certain operations as described herein. In an example, the software can reside (1) on a non-transitory machine readable medium or (2) in a transmission signal. In an example, the software, when executed by the underlying hardware of the circuit, causes the circuit to perform the certain operations.

In an example, a circuit can be implemented mechanically or electronically. For example, a circuit can comprise dedicated circuitry or logic that is specifically configured to perform one or more techniques such as discussed above, such as including a special-purpose processor, a field programmable gate array (FPGA) or an application-specific integrated circuit (ASIC). In an example, a circuit can comprise programmable logic (e.g., circuitry, as encompassed within a general-purpose processor or other programmable processor) that can be temporarily configured (e.g., by software) to perform the certain operations. It will be appreciated that the decision to implement a circuit mechanically (e.g., in dedicated and permanently configured circuitry), or in temporarily configured circuitry (e.g., configured by software) can be driven by cost and time considerations.

Accordingly, the term “circuit” is understood to encompass a tangible entity, be that an entity that is physically constructed, permanently configured (e.g., hardwired), or temporarily (e.g., transitorily) configured (e.g., programmed) to operate in a specified manner or to perform specified operations. In an example, given a plurality of temporarily configured circuits, each of the circuits need not be configured or instantiated at any one instance in time. For example, where the circuits comprise a general-purpose processor configured via software, the general-purpose processor can be configured as respective different circuits at different times. Software can accordingly configure a processor, for example, to constitute a particular circuit at one instance of time and to constitute a different circuit at a different instance of time.

In an example, circuits can provide information to, and receive information from, other circuits. In this example, the circuits can be regarded as being communicatively coupled to one or more other circuits. Where multiple of such circuits exist contemporaneously, communications can be achieved through signal transmission (e.g., over appropriate circuits and buses) that connect the circuits. In embodiments in which multiple circuits are configured or instantiated at different times, communications between such circuits can be achieved, for example, through the storage and retrieval of information in memory structures to which the multiple circuits have access. For example, one circuit can perform an operation and store the output of that operation in a memory device to which it is communicatively coupled. A further circuit can then, at a later time, access the memory device to retrieve and process the stored output. In an example, circuits can be configured to initiate or receive communications with input or output devices and can operate on a resource (e.g., a collection of information).

The various operations of method examples described herein can be performed, at least partially, by one or more processors that are temporarily configured (e.g., by software) or permanently configured to perform the relevant operations. Whether temporarily or permanently configured, such processors can constitute processor-implemented circuits that operate to perform one or more operations or functions. In an example, the circuits referred to herein can comprise processor-implemented circuits.

Similarly, the methods described herein can be at least partially processor-implemented. For example, at least some of the operations of a method can be performed by one or processors or processor-implemented circuits. The performance of certain of the operations can be distributed among the one or more processors, not only residing within a single machine, but deployed across a number of machines. In an example, the processor or processors can be located in a single location (e.g., within a home environment, an office environment or as a server farm), while in other examples the processors can be distributed across a number of locations.

The one or more processors can also operate to support performance of the relevant operations in a “cloud computing” environment or as a “software as a service”

(SaaS). For example, at least some of the operations can be performed by a group of computers (as examples of machines including processors), with these operations being accessible via a network (e.g., the Internet) and via one or more appropriate interfaces (e.g., Application Program Interfaces (APIs).)

Example embodiments (e.g., apparatus, systems, or methods) can be implemented in digital electronic circuitry, in computer hardware, in firmware, in software, or in any combination thereof. Example embodiments can be implemented using a computer program product (e.g., a computer program, tangibly embodied in an information carrier or in a machine readable medium, for execution by, or to control the operation of, data processing apparatus such as a programmable processor, a computer, or multiple computers).

A computer program can be written in any form of programming language, including compiled or interpreted languages, and it can be deployed in any form, including as a stand-alone program or as a software module, subroutine, or other unit suitable for use in a computing environment. A computer program can be deployed to be executed on one computer or on multiple computers at one site or distributed across multiple sites and interconnected by a communication network.

In an example, operations can be performed by one or more programmable processors executing a computer program to perform functions by operating on input data and generating output. Examples of method operations can also be performed by, and example apparatus can be implemented as, special purpose logic circuitry (e.g., a field programmable gate array (FPGA) or an application-specific integrated circuit (ASIC)).

The computing system can include clients and servers. A client and server are generally remote from each other and generally interact through a communication network. The relationship of client and server arises by virtue of computer programs running on the respective computers and having a client-server relationship to each other. In embodiments deploying a programmable computing system, it will be appreciated that both hardware and software architectures require consideration. Specifically, it will be appreciated that the choice of whether to implement certain functionality in permanently configured hardware (e.g., an ASIC), in temporarily configured hardware (e.g., a combination of software and a programmable processor), or a combination of permanently and temporarily configured hardware can be a design choice. Below are set out hardware (e.g., machine 400) and software architectures that can be deployed in example embodiments.

In an example, the machine 400 can operate as a standalone device or the machine 400 can be connected (e.g., networked) to other machines.

In a networked deployment, the machine 400 can operate in the capacity of either
a server or a client machine in server-client network environments. In an example, machine 400 can act as a peer machine in peer-to-peer (or other distributed) network environments. The machine 400 can be a personal computer (PC), a tablet PC, a set-top box (STB), a Personal Digital Assistant (PDA), a mobile telephone, a web appliance, a network router, switch or bridge, or any machine capable of executing instructions
(sequential or otherwise) specifying actions to be taken (e.g., performed) by the machine 400. Further, while only a single machine 400 is illustrated, the term “machine” shall also be taken to include any collection of machines that individually or jointly execute a set (or multiple sets) of instructions to perform any one or more of the methodologies discussed herein.

Example machine (e.g., computer system) 400 can include a processor 402 (e.g., a central processing unit (CPU), a graphics processing unit (GPU) or both), a main memory 404 and a static memory 406, some or all of which can communicate with each other via a bus 408. The machine 400 can further include a display unit 410, an alphanumeric input device 412 (e.g., a keyboard), and a user interface (UI) navigation device 411 (e.g., a mouse). In an example, the display unit 810, input device 417 and UI navigation device 414 can be a touch screen display. The machine 400 can additionally include a storage device (e.g., drive unit) 416, a signal generation device 418 (e.g., a speaker), a network interface device 420, and one or more sensors 421, such as a global positioning system
(GPS) sensor, compass, accelerometer, or other sensor.

The storage device 416 can include a machine readable medium 422 on which is stored one or more sets of data structures or instructions 424 (e.g., software) embodying or utilized by any one or more of the methodologies or functions described herein. The instructions 424 can also reside, completely or at least partially, within the main memory 404, within static memory 406, or within the processor 402 during execution thereof by the machine 400. In an example, one or any combination of the processor 402, the main memory 404, the static memory 406, or the storage device 416 can constitute machine readable media.

While the machine readable medium 422 is illustrated as a single medium, the term “machine readable medium” can include a single medium or multiple media (e.g., a centralized or distributed database, and/or associated caches and servers) that configured to store the one or more instructions 424. The term “machine readable medium” can also be taken to include any tangible medium that is capable of storing, encoding, or carrying instructions for execution by the machine and that cause the machine to perform any one or more of the methodologies of the present disclosure or that is capable of storing, encoding or carrying data structures utilized by or associated with such instructions. The term “machine readable medium” can accordingly be taken to include, but not be limited to, solid-state memories, and optical and magnetic media. Specific examples of machine readable media can include non-volatile memory, including, by way of example, semiconductor memory devices (e.g., Electrically Programmable Read-Only Memory
(EPROM), Electrically Erasable Programmable Read-Only Memory (EEPROM)) and flash memory devices; magnetic disks such as internal hard disks and removable disks; magneto-optical disks; and CD-ROM and DVD-ROM disks.

The instructions 424 can further be transmitted or received over a communications network 426 using a transmission medium via the network interface device 420 utilizing any one of a number of transfer protocols (e.g., frame relay, IP, TCP, UDP, HTTP, etc.). Example communication networks can include a local area network (LAN), a wide area network (WAN), a packet data network (e.g., the Internet), mobile telephone networks (e.g., cellular networks), Plain Old Telephone (POTS) networks, and wireless data networks (e.g., IEEE 802.11 standards family known as Wi-Fi®, IEEE 802.16 standards family known as WiMax®), peer-to-peer (P2P) networks, among others. The term “transmission medium” shall be taken to include any intangible medium that is capable of storing, encoding or carrying instructions for execution by the machine, and includes digital or analog communications signals or other intangible medium to facilitate communication of such software.

Additional aspects of this disclosure are shown in the claims that follow this detailed description

### REFERENCES

[1] Boulbaba Ben Amor, Jingyong Su, and Anuj Srivastava. 2015. Action recognition using rate-invariant analysis of skeletal shape trajectories. IEEE transactions on pattern analysis and machine intelligence 38, 1 (2015), 1-13.
[2] Michael H Brooke, Robert C Griggs, Jerry R Mendell, Gerald M Fenichel, Jack B Shumate, and Richard J Pellegrino. 1981. Clinical trial in Duchenne Dystrophy. I. The design of the protocol. Muscle & Nerve 4, 3 (5 1981), 186-197. https://doi.org/10.1002/mus.880040304.
[3] H Choi, Q Wang. M Toledo, P Turaga, M Buman, and A Srivastava. 2018. Temporal Alignment Improves Feature Quality: An Experiment on Activity Recognition with Accelerometer Data. In 2018 IEEE/CVF Conference on Computer Vision and Pattern Recognition Workshops (CVPRW). 462-4628. https://doi.org/10.1109/CVPRW.2018.00075.
[4] P Thomas Fletcher and Sarang Joshi. 2007. Riemannian geometry for the statistical analysis of diffusion tensor data. Signal Processing 87, 2 (2007), 250-262.
[5] Julaine M Florence, Shree Pandya, Wendy M King, Jenny Diveley Robison, Linda C Signore, Mindy Wentzell, and Michael A Province. 1984. Clinical trials in Duchenne dystrophy: standardization and reliability of evaluation procedures. Physical therapy 64, 1 (1984), 41-45.
[6] A M Glanzman, E Mazzone, M Main, M Pelliccioni, J Wood, K J Swoboda, C Scott, M Pane, S Messina, E Bertini, E Mercuri, and R S Finkel. 2010. The Children's Hospital of Philadelphia Infant Test of Neuromuscular Disorders (CHOP INTEND): test development and reliability. Neuromuscular disorders: NMD 20, 3 (3 2010), 155-161. https://doi.org/10.1016/j.nmd.2009.11.014.
[7] David George Kendall, Dennis Barden, Thomas K Carne, and Huiling Le. 2009. Shape and shape theory. John Wiley & Sons.
[8] Eric Klassen, Anuj Srivastava, M Mio, and Shantanu H Joshi. 2004. Analysis of planar shapes using geodesic paths on shape spaces. IEEE transactions on pattern analysis and machine intelligence 26, 3 (2004), 372-383.
[9] Eric W Ottesen. 2017. ISS-N1 makes the first FDA-approved drug for spinal muscular atrophy. Translational neuroscience 8, 1 (2017), 1-6.
[10] D Ramsey, M Scoto, A Mayhew, M Main, E S Mazzone, J Montes, R de Sanctis, S Dunaway Young, R Salazar, A M Glanzman, A Pasternak, J Quigley, E Mirek, T Duong, R Gee, M Civitello, G Tennekoon, M Pane, M C Pera, K Bushby, J Day, B T Darras, D De Vivo, R Finkel, E Mercuri, and F Muntoni. 2017. Revised Hammersmith Scale for spinal muscular atrophy: A SMA specific clinical outcome assessment tool. PLOS One 12, 2 (2017), e0172346. https://doi.org/10.1371/journal. pone.0172346.
[11] J Derek Tucker, Wei Wu, and Anuj Srivastava. 2013. Generative models for functional data using phase and amplitude separation. Computational Statistics & Data Analysis 61 (2013), 50-66.

1. A computerized system for calculating a respective composite index for respective sets of functional movement scores that quantify motor function abilities for patients diagnosed with a disease,
a wearable sensor positioned proximately to a test patient's anatomy to gather test motion shape data and test motion symmetry data;
a respective wearable sensor positioned proximately to a control patient's anatomy to gather control motion shape data and control motion symmetry data;
a kinetic energy sensor positioned proximately to a test patient's anatomy to gather test kinetic energy data;
a respective control kinetic energy sensor positioned proximately to a control patient's anatomy to gather control kinetic energy data;
a computer comprising software implemented by a computer processor in communication with computer memory, wherein the computer has access to the test motion shape data, the test motion symmetry data, the control motion shape data, the control motion symmetry data, the test kinetic energy data, the control kinetic energy data, and
wherein the software executes computerized steps with the computer processor to calculate and store the composite index from predictors of the functional movement scores, the predictors comprising a respective shape of motion factor, a respective motion symmetry factor, and a respective motion speed factor for each of the sets of functional movement scores, and wherein the computerized steps comprise:
calculating the respective shape of motion factor for each of the test motion shape data and the control motion shape data;
calculating the respective motion symmetry factor for each of the test motion symmetry data and the control motion symmetry data;
calculating the respective motion speed factor for each of the test kinetic energy data and the control kinetic energy data; and
using the software to tabulate the respective composite index by calculating a probability that the respective shape of motion factor, the respective motion symmetry factor, and the respective motion speed factor correspond to one of the sets of functional movement scores.

2. The computerized system of claim 1, wherein calculating the probability comprises assigning weights to the predictors and running the predictors through a regression analysis using machine learning software.

3. The computerized system of claim 1, further comprising:
an imaging device producing images of muscles of the test patient during motion assessment exercises; and
a dynamometer gathering measurements of force exerted by the muscles during the motion assessment exercise, wherein the computer receives the images and the dynamometer measurements and classifies corresponding muscle scores for the muscles; and
wherein the software uses the corresponding muscle scores as an additional predictor in tabulating the composite index.

4. The computerized system of claim 3, wherein the imaging device comprises a linear array ultrasound probe that images a transverse plane and a longitudinal plane of the muscles.

5. The computerized system of claim 3, wherein the images comprise muscle measurements comprising anatomical cross sectional area (ACSA), muscle thickness, and tissue echogenicity.

6. The computerized system of claim 3, wherein the computer classifies corresponding muscle scores by calculating an anatomical cross sectional area (ACSA) of the muscles and dividing the ACSA by an average echogenicity of the muscle.

7. The computerized system of claim 1, wherein the wearable sensor and the respective wearable sensor each comprise a multimodal accelerometer.

8. The computerized system of claim 7, wherein the wearable sensor and the respective wearable sensor each further comprise a gyroscope.

9. The computerized system of claim 1, wherein the wearable sensor and the respective wearable sensor each comprise a wireless sensor in electronic communication with the computer.

10. The computerized system of claim 1, wherein the kinetic energy sensor and the respective control kinetic energy sensor each comprise a manometer measuring forearm pronation and bicep curl motion.

11. The computerized system of claim 1, further comprising a video camera recording video and audio data of the test patient during motion assessment exercises.

12. A computer implemented method of assessing body movements performed by a test patient diagnosed with a disease, the method comprising:
gathering test motion data for the test patient with a sensor positioned proximately to the test patient's anatomy;
gathering control motion data for a control patient with a respective sensor positioned proximately to the control patient's anatomy;
storing, in computer memory connected to a computer processor, test motion data trajectories and control motion data trajectories for respective sets of the test motion data and the control motion data;
using software accessible by the computer processor and the computer memory to perform computer automated steps comprising:
aligning the control motion data trajectories in the time domain;
calculating a mean motion data trajectory for the control motion data trajectories;
aligning the test motion data trajectories to the mean motion data trajectory in the time domain; and
computing distance measurements between the test motion data trajectories and the mean motion data trajectory to quantify severity of motor function symptoms of the disease in the test patient.

13. The computer implemented method of claim 12, wherein computing distances comprises computing at least one of an amplitude distance, a phase distance, or a cosine distance between the test motion data trajectories and the mean motion data trajectories.

14. The computer implemented method of claim 13, wherein computing distances comprises computing a phase distance by computing a test motion data warping function to align the test motion data trajectories to the mean motion data trajectory.

15. The computer implemented method of claim 12, further comprising, prior to aligning the control motion data trajectories, calculating the square root velocity function of the test motion data trajectories and the control motion data trajectories.

16. The computer implemented method of claim 15, further comprising computing the distance measurements in the time domain across a range of values for the square root velocity functions of the test motion data trajectories to the mean motion data trajectory.

17. The computer implemented method of claim 12, further comprising using the software to complete phase amplitude separation on the test motion data trajectories and the control motion trajectories prior to calculating the mean motion data trajectory.

18. The computer implemented method of claim 12, wherein gathering the test motion data and the control motion data comprises attaching the sensor to the test patient's anatomy and attaching the respective sensor to the control patient's anatomy.

19. The computer implemented method of claim 12, wherein gathering the test motion data and the control motion data comprises gathering data with a camera.

20. The computer implemented method of claim 12, further comprising gathering muscle structure image data from the test patient and correlating the muscle structure image data with the distance measurements in the time domain.

21.-25. (canceled)
